(19) United States
(12) Patent Application Publication
(10) Pub. No.: US 2025/0268892 A1
(43) Pub. Date: Aug. 28, 2025

(54) TREATMENT OF CANCER USING ARIPIPRAZOLE AND ANTICANCER AGENT

(71) Applicant: VSPharm Tech Co., Ltd., Daegu (KR)

(72) Inventors: Shin Young Park, Seoul (KR); Hyunho Lee, Seoul (KR); SoHee Kim, Gyeonggi-do (KR); Eun-Ran Park, Seoul (KR); Soon-Sun Hong, Seoul (KR); Kyung Hee Jung, Incheon (KR); Yejin Cho, Incheon (KR)

(21) Appl. No.: 19/058,376

(22) Filed: Feb. 20, 2025

Related U.S. Application Data
(60) Provisional application No. 63/558,188, filed on Feb. 27, 2024.

(51) Int. Cl. A61K 31/496; A61K 31/282; A61K 33/243; A61P 35/00

(52) U.S. Cl. CPC A61K 31/496; A61K 31/282; A61K 33/243; A61P 35/00

(57) ABSTRACT

Aripiprazole, a compound typically used for psychiatric conditions, enhances the impact of various anticancer agents on pancreatic, glioma, colorectal, and lung cancers. By lowering levels of anti-apoptotic proteins (e.g., XIAP, MCL-1, cFLIP) and interfering with STAT3 signaling, it intensifies apoptotic pathways and counters drug resistance. This combined approach suppresses tumor growth without significant added toxicity, offering a more potent and better-tolerated treatment option for aggressive malignancies.

### CROSS-REFERENCE TO RELATED APPLICATIONS

This application is based on and claims priority to U.S. Provisional Application Ser. No. 63/558,188, filed on Feb. 27, 2024, which is incorporated herein by reference in its entirety.

### FIELD OF THE INVENTION

The present invention generally relates to a method of treating cancer in a patient in need thereof by conjointly administering aripiprazole and an anticancer agent to the patient. The present invention is also generally related to a newly discovered mechanism behind the cancer treatment.

### BACKGROUND OF THE INVENTION

According to data released by the American Cancer Society, 17% (7.6 million) of worldwide deaths in 2008 were due to cancer, and the number of deaths due to cancer is expected to surge to 9 million in 2015 and 11.4 million in 2030. The number one among 10 causes of death is cancer, and the number of deaths from cancer per 100,000 people is 153 and has been on the rise every year. As a result, the social costs are huge, and the need for the development of less toxic and more effective anti-cancer drugs has gradually increased. In addition, recently, due to the huge social costs of cancer, the development of drugs that can be treated at a low cost is required.

All types of cancer have been commonly treated with chemotherapy, however due to drug resistance, its effectiveness is frequently reduced or totally eliminated. For instance, Wang (2014) reports that over 90% of mortality in individuals with metastatic breast cancer are caused by treatment resistance. Several potential mechanisms are listed in a recent summary of medication resistance to cancer agents: target expression was elevated, there was MLH1 hypermethylation, activation of survival pathways, increased expression of anti-apoptotic proteins, decreased cellular uptake, increased efflux, increased DNA repair, decreased target expression, topoisomerase I mutations, suppression of apoptosis, MDR1 overexpression, mutation or decreased expression of topoisomerase II; and decreased apoptosis due to mutation of. Efforts to combat the resistance to cancer agents have met with limited success, and usually have involved combinations of anticancer drugs, such as combinations of drugs with different mechanisms, or combinations with drugs that inhibit efflux transporters, such as Pgp.

It would be ideal to develop new therapeutic drugs that are specifically targeted at tumor cells, minimize or eliminate systemic toxicity, and address or avoid drug resistance.

### SUMMARY OF THE INVENTION

The development of new anticancer drugs continues to increase due to the low efficacy of existing anticancer drugs, fatal side effects compared to other treatments, and problems with drug resistance.

Since 2010, the cancer treatment rate for all chemotherapy patients has been stagnant, and efficient new chemotherapy technology is needed.

Aripiprazole is a pre-authorized drug ingredient used in clinical practice as a treatment for schizophrenia and depression.

In the present invention, aripiprazole is expected to be used in combination with existing anticancer drugs to increase the effectiveness of chemotherapy and can be expected in combination with other treatments such as surgery and radiation therapy, so the socioeconomic burden can be minimized by improving the treatment rate more effectively.

Cisplatin, a platinum-based drug, is effective in chemotherapy for various solid tumors such as testicular cancer, head and neck cancer, ovarian cancer, cervical cancer, and non-small cell lung cancer. Carboplatin, another platinum-based drug, and other types of anticancer drugs are administered together to show a certain level of anticancer effect. Oxaliplatin, another platinum-based drug, is a third-generation cisplatin analog and is widely used as an anticancer drug for colon cancer.

Gemcitabine, a chemical drug, has been established as a standard treatment for primary chemotherapy since 1997 and has been a representative pancreatic cancer and lung cancer treatment used as a combination of various treatments and treatments. For pancreatic cancer treatment, however, the average survival time by Gemcitabine alone treatment is about several months, and less than 10% of patients are reported to respond to Gemcitabine. Combination therapy with other anticancer drugs has been tried but has not shown any breakthrough results. 5-FU, another chemical drug, is widely used as an anticancer drug for pancreatic and colorectal cancer and has been the basis of pancreatic and colorectal cancer chemotherapy for the past 60 years. Temozolomide, another chemical drug, exhibits a real anti-tumor effect with low toxicity in glioblastoma patients, and temozolomide chemotherapy for malignant glioma is more effective and safer than previous chemotherapy.

The present invention is in some embodiments related to a method of treating cancer in a patient in need thereof. The method may include conjointly administering aripiprazole and an anticancer agent to the patient, thereby treating the cancer. Conjointly administering aripiprazole and the anticancer agent may provide a synergistic effect. The cancer may be selected from the group consisting of lung cancer, colorectal cancer, pancreatic cancer, and glioma. The anticancer agent may be selected from the group consisting of cisplatin, carboplatin, oxaliplatin, gemcitabine, 5-FU, and temozolomide. In particular, the anticancer agent may be cisplatin.

In some embodiments, a method of treating pancreatic cancer in a patient in need thereof is provided. The method may include conjointly administering aripiprazole and an anticancer agent to the patient, thereby treating the pancreatic cancer. Conjointly administering aripiprazole and the anticancer agent may provide a synergistic effect. The anticancer agent may be selected from the group consisting of cisplatin, carboplatin, oxaliplatin, gemcitabine, 5-FU, and temozolomide. In particular, the anticancer agent may be cisplatin.

In some embodiments, a method of treating glioma in a patient in need thereof is provided. The method may include conjointly administering aripiprazole and an anticancer agent to the patient, thereby treating the glioma. Conjointly administering aripiprazole and the anticancer agent may provide a synergistic effect. The anticancer agent may be selected from the group consisting of cisplatin, carboplatin, oxaliplatin, gemcitabine, 5-FU, and temozolomide. In particular, the anticancer agent may be cisplatin.

In some embodiments, a method of treating colorectal cancer in a patient in need thereof is provided. The method may include conjointly administering aripiprazole and an anticancer agent to the patient, thereby treating the colorectal cancer. Conjointly administering aripiprazole and the anticancer agent may provide a synergistic effect. The anticancer agent may be selected from the group consisting of cisplatin, carboplatin, oxaliplatin, gemcitabine, 5-FU, and temozolomide. The anticancer agent may be carboplatin.

In some embodiments, a method of treating lung cancer in a patient in need thereof is provided. The method may include conjointly administering aripiprazole and an anticancer agent to the patient, thereby treating the lung cancer. Conjointly administering aripiprazole and the anticancer agent may provide a synergistic effect. The anticancer agent may be selected from the group consisting of cisplatin, carboplatin, oxaliplatin, gemcitabine, 5-FU, and temozolomide.

### DESCRIPTION OF DRAWINGS

FIG. 1A represents the level of synergism and antagonism determined by a range of combination index.

FIG. 1B represents three different effects of two different drugs: antagonistic effect, additive effect, and synergistic effect.

FIG. 2A represents the cell viability of PANC-1 as a function of the concentration of aripiprazole and the combination (aripiprazole and cisplatin).

FIG. 2B represents the cell viability of PANC-1 as a function of the concentration of cisplatin.

FIG. 2C represents the synergism of aripiprazole and cisplatin in treating PANC-1 cell line, which is determined by the combination index.

FIG. 3A represents the cell viability of HPAC as a function of the concentration of aripiprazole and the combination (aripiprazole and cisplatin).

FIG. 3B represents the cell viability of HPAC as a function of the concentration of cisplatin.

FIG. 3C represents the synergism of aripiprazole and cisplatin in treating HPAC cell line, which is determined by the combination index.

FIG. 4A represents the cell viability of U-251 MG as a function of the concentration of aripiprazole and the combination (aripiprazole and cisplatin).

FIG. 4B represents the cell viability of U-251 MG as a function of the concentration of cisplatin.

FIG. 4C represents the synergism of aripiprazole and cisplatin in treating U-251 MG cell line, which is determined by the combination index.

FIG. 5A represents the cell viability of U-87 MG as a function of the concentration of aripiprazole and the combination (aripiprazole and cisplatin).

FIG. 5B represents the cell viability of U-87 MG as a function of the concentration of cisplatin.

FIG. 5C represents the synergism of aripiprazole and cisplatin in treating U-87 MG cell line, which is determined by the combination index.

FIG. 6A represents the cell viability of HCT-116 as a function of the concentration of aripiprazole and the combination (aripiprazole and carboplatin).

FIG. 6B represents the cell viability of HCT-116 as a function of the concentration of carboplatin.

FIG. 6C represents the synergism of aripiprazole and cisplatin in treating HCT-116 cell line, which is determined by the combination index.

FIG. 7A represents the cell viability of PANC-1 as a function of the concentration of aripiprazole and the combination (aripiprazole and gemcitabine).

FIG. 7B represents the cell viability of PANC-1 as a function of the concentration of gemcitabine.

FIG. 7C represents the lack of synergism of aripiprazole and gemcitabine in treating PANC-1 cell line, which is determined by the combination index.

FIG. 8A represents the cell viability of the pancreatic cancer cell lines by aripiprazole.

FIG. 8B represents the cell viability of the pancreatic cancer cell lines by anticancer drugs.

FIGS. 8C-8D represent the anticancer efficacy of the combination of aripiprazole and anticancer drugs in MIA PaCa-2 cells.

FIG. 8E represents the anticancer efficacy of the combination of aripiprazole and anticancer drugs in MIA PaCa-2 and Capan-1 cells.

FIG. 8F represents the statistical significance in the cell viability of MIA PaCa-2 and Capan-1 cells by control, aripiprazole and cisplatin, and the combination of aripiprazole and cisplatin.

FIG. 9A represents apoptosis related markers in MIA PaCa-2 and Capan-1 cells after the treatment with control, aripiprazole and cisplatin, and the combination of aripiprazole and cisplatin.

FIG. 9B represents the result of TUNEL assay for MIA PaCa-2 cells and Capan-1 cells after the treatment with control, aripiprazole and cisplatin, and the combination of aripiprazole and cisplatin.

FIGS. 9C and 9D represents the result of Immunocytochemistry (ICC) for MIA PaCa-2 cells and Capan-1 cells after the treatment with control, aripiprazole and cisplatin, and the combination of aripiprazole and cisplatin.

FIGS. 9E and 9F represent the result of JC-1 staining for MIA PaCa-2 cells and Capan-1 cells after the treatment with control, aripiprazole and cisplatin, and the combination of aripiprazole and cisplatin.

FIGS. 10A and 10B represent the result of phospho-kinase array in MIA PaCa-2 cells after 2 hours and 6 hours of the treatment with aripiprazole, respectively.

FIG. 10C represents the result of Western blotting in MIA PaCa-2 and Capan-1 cells after the treatment with control, aripiprazole and cisplatin, and the combination of aripiprazole and cisplatin.

FIG. 10D represents the result of Western blotting in MIA PaCa-2 cells after the treatment of 48 hours, 6 hours, and 2 hours, respectively, with control, aripiprazole and cisplatin, and the combination of aripiprazole and cisplatin.

FIG. 11A represents real-time cell proliferation curves over a 96-hour period, comparing control (CON), cisplatin, ARI, and CB. Both cell lines display significantly lower proliferation in the combination treatment group than in the single-agent or control groups.

FIG. 11B represents colony formation assays for MIA PaCa-2 and Capan-1 cells under CON, cisplatin, ARI, and CB. The images (up) and bar graphs (down) illustrate that the combination treatment results in substantially fewer and smaller colonies compared to the individual treatments, indicating enhanced anticancer potency.

FIG. 12A represents migration assays of MIA PaCa-2 and Capan-1 cells under four conditions (CON, cisplatin, ARI, combination). Fewer migrated cells appear in the combination group.

FIG. 12B represents invasion assays using similar conditions, also demonstrating the strongest inhibition with the combination treatment.

FIG. 12C represents schematic of the fluid shear stress assay used to measure cell adhesion and subsequent spheroid formation. The bar graph shows that combination treatment lowers cell adhesion more than single agents.

FIG. 12D represents representative images of 3D tumor spheroids (under the same treatments). Spheroid size is significantly reduced in the combination group compared to single-agent treatments.

FIG. 13A represents xenograft study in mice bearing MIA PaCa-2 tumors. Photographs (up) and growth curves (down) show the strongest tumor suppression in the combination (CB) group.

FIG. 13B represents orthotopic pancreatic tumor model (KPC cells). Tumor weights indicate similar enhanced suppression by CB.

FIG. 13C represents hematoxylin and eosin staining and immunohistochemical detection analysis (H&E, Ki-67, TUNEL, Bcl-2, cleaved caspase-3, and p-STAT3). Combination-treated tumors have reduced proliferation (Ki-67), increased apoptosis (TUNEL, cleaved caspase-3), and markedly lowered p-STAT3 expression.

FIG. 14 illustrates the antitumor activity observed in different treatment groups using a U87-MG-luc orthotopic model for the efficacy study.

FIG. 15 illustrates how cisplatin plus aripiprazole collectively reduces mitochondrial membrane potential, downregulate XIAP and Mcl-1, inhibit STAT3 signaling, and thereby enhance caspase-mediated apoptosis while suppressing tumor cell proliferation.

FIG. 16 presents a schematic overview of the DNA Damage Response (DDR), including major repair pathways and key regulatory proteins (ATM, ATR, PARP, etc.).

FIG. 17 illustrates how chemotherapeutic or radiation-induced DNA breaks increase γ-H2AX and drive cell-fate decisions toward apoptosis when damage is irreparable.

FIG. 18 shows how cFLIP inhibition and enhanced DNA damage jointly promote extrinsic apoptosis, activating caspase-8 and its downstream effectors.

FIG. 19 demonstrates DNA fragmentation in multiple cancer cell lines treated with aripiprazole alone or combined with ionizing radiation, indicating heightened cell death.

FIG. 20 depicts sustained DNA damage responses via Western blot, where co-treatment of aripiprazole and γ-IR elevates γ-H2AX and PARP cleavage more than single agents.

FIG. 21 employs immunocytochemistry in MDA-MB-231 cells to visualize increased γ-H2AX foci under aripiprazole plus radiation, confirming robust DNA damage.

FIG. 22 reinforces the amplified DNA damage in MDA-MB-231 cells by showing enhanced γ-H2AX staining and Western blot data following aripiprazole+IR treatment.

FIG. 23 highlights the downregulation of cFLIP and activation of extrinsic apoptosis (caspase-8) in DU-145 cells with aripiprazole and γ-IR.

FIG. 24 shows reduced cFLIP in lung (A549, H1299) and head and neck (FaDu) cancer cells after combined aripiprazole and γ-IR exposure, enhancing apoptotic signaling.

FIG. 25 demonstrates similar FLIP (L/S) suppression in pancreatic (MIA-PaCa-2, PANC-1) and breast (MCF-7, MDA-MB-231) cells under aripiprazole plus γ-IR.

FIG. 26 confirms elevated DNA damage markers (γ-H2AX, PARP cleavage) and diminished cFLIP in breast and prostate cancer lines after aripiprazole and radiation co-treatment.

### DETAILED DESCRIPTION OF THE PREFERRED EMBODIMENTS

Further in relation to this, before explaining at least the preferred embodiments of the invention in greater detail, it is to be understood that the invention is not limited in its application to the details of construction and to the arrangements of the components set forth in the following description. It would be understood by those of ordinary skill in the art that embodiments beyond those described herein are contemplated, and the embodiments can be practiced and carried out in a plurality of different ways. Also, it is to be understood that the terminology used herein is for the purpose of description and should not be regarded as a limiting factor.

Unless otherwise defined, the terms used herein refer to that which the ordinary artisan would understand such term to mean based on the contextual use of such term herein. To the extent that the meaning of a term used herein as understood by the ordinary artisan based on the contextual use of such term differs in any way from any particular dictionary definition of such term, it is intended that the meaning of the term as understood by the ordinary artisan will prevail.

As used herein, the phrase “conjoint administration” or its derivation refers to any form of administration of two or more different therapeutic compounds such that the second compound is administered while the previously administered therapeutic compound is still effective in the body (e.g., the two compounds are simultaneously effective in the patient, which may include synergistic effects of the two compounds).

As used herein, the term “about” means approximately or nearly and in the context of a numerical value or range set forth herein means 10% of the numerical value or range recited or claimed.

As used herein, the term “treating” includes delaying, alleviating, mitigating or reducing the intensity, progression, or worsening of one or more attendant symptoms of a disorder or condition and/or alleviating, mitigating or impeding one or more causes of a disorder or condition. Treatment under the claimed invention may be a preventative treatment, prophylactic treatment, remission of treating or ameliorating treatment.

As used herein, “consists essentially of” when used in conjunction with a composition means excluding other materials that do not materially contribute to treating cancer. With the language, other materials that contribute to the treatment that materially affect the basic and novel characteristics of the disclosure are not required and are potentially counterproductive because they may offset the treatment effect of aripiprazole and anticancer agent. Small traces that have little or no effect to the treatment as part of the embodiments of the presentation disclosure may exist in a composition that consists essentially of aripiprazole and anticancer agent under the definition because it would not materially affect its function and/or objective.

As used herein, the term “patient”, as used herein, refers to a human or non-human animal such as a primate, non-human primate, laboratory animal, farm animal, livestock, or a domestic pet.

As used herein, the term “anticancer agent” refers to any agent that exhibits anti-tumor activity. By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a stabilization or decrease in the overall size of a tumor during therapy.

As used herein, the term “aripiprazole” refers to 7-[4-[4-(2,3-Dichlorophenyl)-1-piperazinyl]butoxy]-3,4-dihydro-2 (1H)-quinolinone, its derivatives, solvates, hydrates, its pharmaceutically acceptable salts, and mixtures thereof.

As used herein, the phrase “pharmaceutically acceptable”, as used in connection with compositions of the invention, refers to molecular entities and other ingredients of such compositions that are physiologically tolerable and do not typically produce untoward reactions when administered to a mammal (e.g., human). The term “pharmaceutically acceptable” may also mean approved by a regulatory agency of the Federal or a state government or listed in the U.S. Pharmacopeia or other generally recognized pharmacopeia for use in mammals, and more particularly in humans.

As used herein, the term “salt” is defined as a chemical containing different charged components. The term salt also includes hydrates and solvates. Contemplated in the instant description are pharmaceutically acceptable salts, which salts may include, but are not limited to, acid addition salts, such as those made with hydrochloric, sulfuric, nitric, phosphoric, acetic, maleic, fumaric, tartaric, citric, benzoic, methane sulphonic, naphthalene sulphonic, p-toluene sulphonic acid. All of these salts (or other similar salts) may be prepared by conventional means. The nature of the salt is not critical, provided that it is non-toxic and does not substantially interfere with the desired pharmacological activity.

As used herein, the term “cisplatin” refers to (SP-4-2)-diamminedichloridoplatinum (II) (Chemical Abstracts Services Registry No. 15663-27-1).

As used herein, the term “carboplatin” refers to a composition comprising the anticancer agent identified by the Chemical Abstracts registry number 41575-94-4.

As used herein, the term “oxaliplatin” refers to a composition comprising the anticancer agent identified by the Chemical Abstracts registry number 61825-94-3.

As used herein, the term “gemcitabine” refers to a composition comprising the anticancer agent identified by the Chemical Abstracts registry number 95058-81-4.

As used herein, the term “5-FU” refers to a composition comprising the anticancer agent identified by the Chemical Abstracts registry number 51-21-8.

As used herein, the term “temozolomide” means a compound having the chemical name 4-methyl-5-oxo-2, 3,4,6, 8-pentazabicyclo [4.3.0] nonane-2, 7, 9-triene-9-carboxamide and having the chemical abstracts registry number 85622-93-1.

The term “pancreatic cancer” or “pancreas cancer” as used herein relates to cancer which is derived from pancreatic cells including but not limited to, adenocarcinomas, adenosquamous carcinomas, signet ring cell carcinomas, hepatoid carcinomas, colloid carcinomas, undifferentiated carcinomas, undifferentiated carcinomas with osteoclast-like giant cells and islet cell carcinomas.

The term “glioma”, as used herein, refers to a type of cancer that starts in the brain or spine and which arises from glial cells and/or its precursors including Ependymomas (gliomas derived from ependymal cells), astrocytomas (gliomas derived from astrocytes and which includes glioblastoma multiforme, oligodendrogliomas, (gliomas derived from oligodendrocytes) and mixed gliomas, such as oligoastrocytomas (derived from cells from different types of glia).

The term “lung cancer”, as used herein, refers to any uncontrolled cell growth in tissues of the lung, including but not limited to, small cell lung carcinoma, combined small cell carcinoma, non-small cell lung carcinoma, sarcomatoid carcinoma, salivary gland tumors, carcinoid tumor, adenosquamous carcinoma, pleuropulmonary blastoma and carcinoid tumor.

As used herein, “colon cancer,” also called “colorectal cancer” or “bowel cancer,” refers to a malignancy that arises in the large intestine (colon) or the rectum (end of the colon), and includes cancerous growths in the colon, rectum, and appendix, including adenocarcinoma.

The term “combination index (CI)” used herein refers to a quantitative measure of the interaction (synergistic, additive, or antagonistic) between two drugs, typically calculated according to the method of Chou-Talalay. A CI value below 1 indicates synergy, a CI equal to 1 indicates an additive effect, and a CI above 1 indicates antagonism.

The term “GI50” used herein refers to “growth inhibition 50,” i.e., the drug concentration at which cell proliferation is reduced by 50%. It is determined by treating cells with increasing concentrations of a compound and then measuring cell viability or growth to identify the dose at which growth is half of that in an untreated control.

The term “MTT assay” used herein refers to a colorimetric assay for assessing cell metabolic activity, where viable cells convert the water-soluble MTT reagent into insoluble formazan crystals. After solubilization, the absorbance of formazan at a specific wavelength (usually ˜540 nm) indicates the number of metabolically active cells.

The term “CellTiter-Glo” used herein refers to a luminescence-based assay that measures cellular ATP content, serving as an indicator of metabolically active (viable) cells. When the reagent lyses cells, the released ATP drives a luciferase reaction, producing light proportional to the number of living cells.

The term “TUNEL assay” used herein refers to the “Terminal deoxynucleotidyl transferase dUTP Nick-End Labeling” assay, which detects DNA fragmentation by labeling free 3′-OH termini, indicating cells undergoing apoptosis. TUNEL-positive nuclei typically signify apoptotic cell death.

The term “JC-1 staining” used herein refers to a fluorescence-based method for assessing mitochondrial membrane potential. In healthy mitochondria with high membrane potential, JC-1 aggregates, emitting red fluorescence, whereas in depolarized mitochondria, it remains in a monomeric form emitting green fluorescence, thereby indicating apoptosis.

The term “transwell migration” (or “transwell invasion”) used herein refers to a modified Boyden chamber assay in which cells migrate or invade through a porous membrane (optionally coated with Matrigel for invasion). The number of cells crossing the membrane under different treatment conditions reveals how the treatment affects cancer cell motility or invasiveness.

The term “colony formation assay” used herein refers to a technique in which cells are seeded at low density, allowed to grow for an extended period (e.g., 14 days), and then stained to visualize colonies. Fewer or smaller colonies after a given treatment reflect reduced proliferative or clonogenic capacity of the cells.

The term “xenograft” used herein refers to an in vivo cancer model in which human cancer cells or tissues are implanted (subcutaneously or orthotopically) into immunocompromised (e.g., nude or SCID) mice to study tumor growth and evaluate therapeutic efficacy under experimental treatments.

The term “orthotopic tumor model” used herein refers to an in vivo model where tumor cells are implanted into the organ or tissue of origin (e.g., pancreatic cancer cells injected into the mouse pancreas). This model more closely mimics the tumor's native microenvironment and metastatic behavior compared to subcutaneous implantation.

### Synergistic Combination of Aripiprazole and Anticancer Agent

In accordance with the principles of the present invention, the use of aripiprazole and anticancer agent to treat cancer is provided. In particular, some embodiments are directed to use of aripiprazole and anticancer agent to treat pancreatic cancer, glioma, colorectal cancer, and lung cancer.

In some embodiments, cells (e.g., PANC-1, HPAC, MIA PaCa-2, AsPC-1 for pancreatic cancer; U-251 MG, U-85 MG glioma; HCT-116 for colorectal cancer; and H460 for lung cancer) were cultured in the incubator at 37° C., 5% CO₂, using 10% Fetal Bovine Serum (FBS; Gibco, Rockville MD, USA) and 1% Antibiotic/Antimycotic solution Dulbecco's Modified Eagle's Medium (DMEM; Gibco) added Roswell Park Memorial Institute (RPMI-1640; Gibco), Dulbecco's Modified Eagle Medium/Nutrient Mixture F-12 (DMEM-F12; Gibco). It should be understood that cells encompassed by the present invention are not limited by the above listed exemplary cells.

For the cell growth inhibition measurement of cell growth, 3,000 cells per well were incubated overnight. The next day, after chemically treating the cells, the cells were cultured for a total of 3 days to obtain the results. After processing CellTiter-Glo Luminescent cell viability assay (Promega), the value of luminescent was obtained using the multiplate reader (Biotek).

To measure the level of synergism, cell growth was quantified as a percentage through the value obtained through CellTiter-Glo Luminescent cell viability assay, which was converted back into effect values.

$$Cell growth ( \% ) = ( Group 1 average - Seeding control ) / ( DMSO or none treated control - Seeding control ) * 100$$

$$Effect = ( 100 - Cell growth , \% ) / 100$$

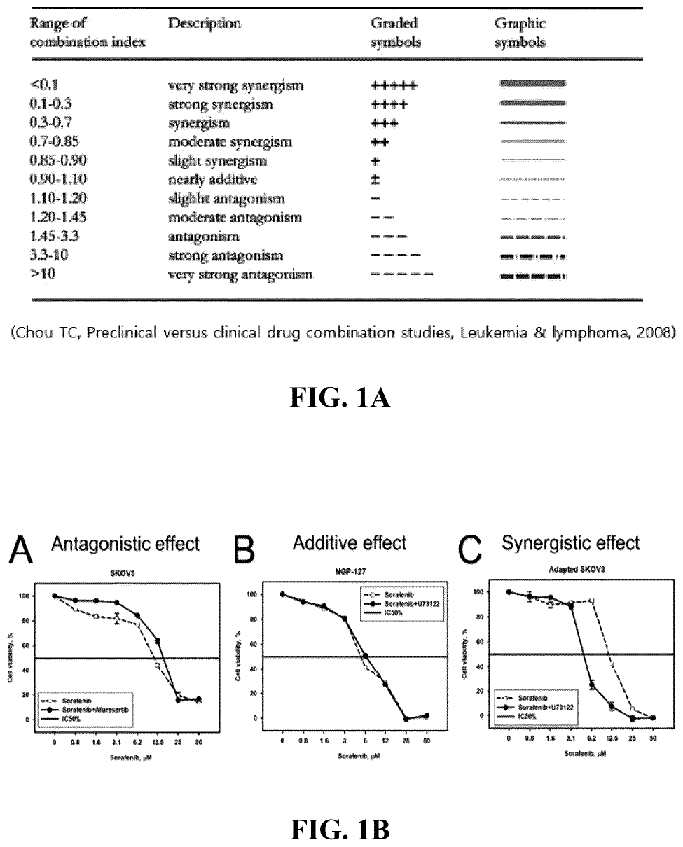

The converted effect value is determined by calculating the effect value according to the drug concentration into the combination index (CI) value through the CalcuSyn (Version 2.11, Biosoft) program, and then the synergism corresponding to each CI value is indicated, as shown in FIG. 1A. More detailed information about determining synergism and measuring CI value by the CI method of Chou-Talay may be found from Chou TC. Preclinical versus clinical drug combination studies. Leuk Lymphoma. 2008, which is hereby incorporated by its entirety.

Generally, the method provides quantitative determination for synergism (CI<1), additive effect (CI=1) and antagonism (CI>1), and provides the algorithm for computer software for automated simulation for drug combinations, as shown in FIG. 1A.

If the CI value is below 0.1, the combination indicates very strong synergism. If the CI value is between 0.1 and 0.3, the combination indicates strong synergism. If the CI value is between 0.3 and 0.7, the combination indicates synergism. If the CI value is between 0.7 and 0.85, the combination indicates moderate synergism. If the CI value is between 0.85 and 0.90, the combination indicates slight synergism. If the CI value is between 0.90 and 1.10, the combination indicates nearly additive. If the CI value is between 1.10 and 1.20, the combination indicates slight antagonism. If the CI value is between 1.20 and 1.45, the combination indicates moderate antagonism. If the CI value is between 1.45 and 3.3, the combination indicates antagonism. If the CI value is between 3.3 and 10, the combination indicates strong antagonism. If the CI value is above 10, the combination indicates very strong antagonism.

As shown in FIG. 1B, further classifications of antagonistic effect, additive effect, and synergistic effect may be found from (Sorokin M et al., Oncobox Bioinformatical Platform for Selecting Potentially Effective Combinations of Target Cancer Drugs Using High-Throughput Gene Expression Data, cancers, 2018), which is hereby incorporated by its entirety.

In some embodiments, synergism may include very strong synergism only. In some embodiments, synergism may include very strong synergism and strong synergism. In some embodiments, synergism may include very strong synergism, strong synergism, and synergism. In some embodiments, synergism may include very strong synergism, strong synergism, synergism, and moderate synergism. In some embodiments, synergism may include very strong synergism, strong synergism, synergism, moderate synergism, and slight synergism.

In some embodiments, antagonism may include very strong antagonism only. In some embodiments, synergism may include very strong antagonism and strong antagonism. In some embodiments, antagonism may include very strong antagonism, strong antagonism, and antagonism. In some embodiments, antagonism may include very strong antagonism, strong antagonism, antagonism, and moderate antagonism. In some embodiments, antagonism may include very strong antagonism, strong antagonism, antagonism, moderate antagonism, and slight antagonism.

For each of the combinations of aripiprazole and anticancer agents (e.g., cisplatin, carboplatin, oxaliplatin, gemcitabine, 5-FU, and temozolomide), GI₅₀ for aripiprazole was determined. GI₅₀, or growth inhibition 50, is the maximum concentration at which the cell proliferation is halved when the drug is administered. GI₅₀ for aripiprazole was determined to establish a range for the concentration of aripiprazole to be tested. It should be understood that anticancer agents encompassed by the present invention are not limited by the above exemplary anticancer agents.

For anticancer agents, a concentration at which about 75˜85% cell viability was determined. In some embodiments, a concentration at which about 75% cell viability may be determined. In some embodiments, a concentration at which about 80% cell viability may be determined. In some embodiments, a concentration at which about 85% cell viability may be determined.

While fixing the determined concentration of anticancer agents, various concentrations in the determined range for the concentration of aripiprazole were tested.

In some embodiments, a method of treating cancer in a patient in need thereof may include conjointly administering aripiprazole and an anticancer agent to the patient, thereby treating the cancer. Conjointly administering aripiprazole and the anticancer agent may provide a synergistic effect. The cancer may be selected from the group consisting of lung cancer, colorectal cancer, pancreatic cancer, and glioma. The anticancer agent may be selected from the group consisting of cisplatin (CAS NO: 15663-27-1), carboplatin (CAS NO: 41575-94-4), oxaliplatin (CAS NO: 61825-94-3), gemcitabine (CAS NO: 95058-81-4), 5-FU (CAS NO: 51-21-8), and temozolomide (CAS NO: 85622-93-1). In some embodiments, any anticancer agent known to be effective for treating cancer may be used. In particular, the anticancer agent may be cisplatin. In some embodiments, more than one anticancer agent may be conjointly administered to a patient with aripiprazole.

In some embodiments, a method of treating cancer in a patient in need thereof may consist essentially of conjointly administering aripiprazole and an anticancer agent to the patient, thereby treating the cancer.

In some embodiments, a method of treating cancer in a patient in need thereof may consist of conjointly administering aripiprazole and an anticancer agent to the patient, thereby treating the cancer.

In some embodiments, a method of treating pancreatic cancer in a patient in need thereof is provided. The method may include conjointly administering aripiprazole and an anticancer agent to the patient, thereby treating the pancreatic cancer. Conjointly administering aripiprazole and the anticancer agent may provide a synergistic effect. The anticancer agent may be selected from the group consisting of cisplatin, carboplatin, oxaliplatin, gemcitabine, 5-FU, and temozolomide. In some embodiments, any anticancer agent known to be effective for treating pancreatic cancer may be used. In particular, the anticancer agent may be cisplatin. In some embodiments, more than one anticancer agent may be conjointly administered to a patient with aripiprazole.

In some embodiments, a method of treating glioma in a patient in need thereof is provided. The method may include conjointly administering aripiprazole and an anticancer agent to the patient, thereby treating the glioma. Conjointly administering aripiprazole and the anticancer agent may provide a synergistic effect. The anticancer agent may be selected from the group consisting of cisplatin, carboplatin, oxaliplatin, gemcitabine, 5-FU, and temozolomide. In some embodiments, any anticancer agent known to be effective for treating glioma may be used. In particular, the anticancer agent may be cisplatin. In some embodiments, more than one anticancer agent may be conjointly administered to a patient with aripiprazole.

In some embodiments, a method of treating colorectal cancer in a patient in need thereof is provided. The method may include conjointly administering aripiprazole and an anticancer agent to the patient, thereby treating the colorectal cancer. Conjointly administering aripiprazole and the anticancer agent may provide a synergistic effect. The anticancer agent may be selected from the group consisting of cisplatin, carboplatin, oxaliplatin, gemcitabine, 5-FU, and temozolomide. In some embodiments, any anticancer agent known to be effective for treating colorectal cancer may be used. In particular, the anticancer agent may be carboplatin. In some embodiments, more than one anticancer agent may be conjointly administered to a patient with aripiprazole.

In some embodiments, a method of treating lung cancer in a patient in need thereof is provided. The method may include conjointly administering aripiprazole and an anticancer agent to the patient, thereby treating the lung cancer. Conjointly administering aripiprazole and the anticancer agent may provide a synergistic effect. The anticancer agent may be selected from the group consisting of cisplatin, carboplatin, oxaliplatin, gemcitabine, 5-FU, and temozolomide. In some embodiments, any anticancer agent known to be effective for treating lung cancer may be used. In some embodiments, more than one anticancer agent may be conjointly administered to a patient with aripiprazole.

Exemplary illustrative embodiments are explained in the following examples.

### Example 1

To measure the efficacy of the treatment of pancreatic cancer using aripiprazole and an anti-cancer agent, PANC-1, pancreatic human cancer cell line, was cultured and tested.

PANC-1 cell line was cultured in a 5% CO₂ incubator at 37° C., using Dulbecco's Modified Eagle's Medium (DMEM; Gibco), Roswell Park Memorial Institute (RPMI-1640; Gibco), and Dulbecco's Modified Eagle Medium/Nutrient Mixture F-12 (DMEM-F12; Gibco) supplemented with 10% Fetal Bovine Serum (FBS; Gibco, Rockville MD, USA) and 1% Antibiotic/Antimycotic solution.

For measuring cell growth inhibition, 3,000 PANC-1 cells per well were incubated overnight. The next day, after chemically treating the cells, the cells were cultured for 3 days to obtain the results. After processing CellTiter-Glo Luminescent cell viability assay (Promega), the value of luminescent was obtained using the multiplate reader (Biotek). To measure the level of synergism, cell growth was quantified as a percentage through the value obtained through CellTiter-Glo Luminescent cell viability assay, which was converted back into effect values.

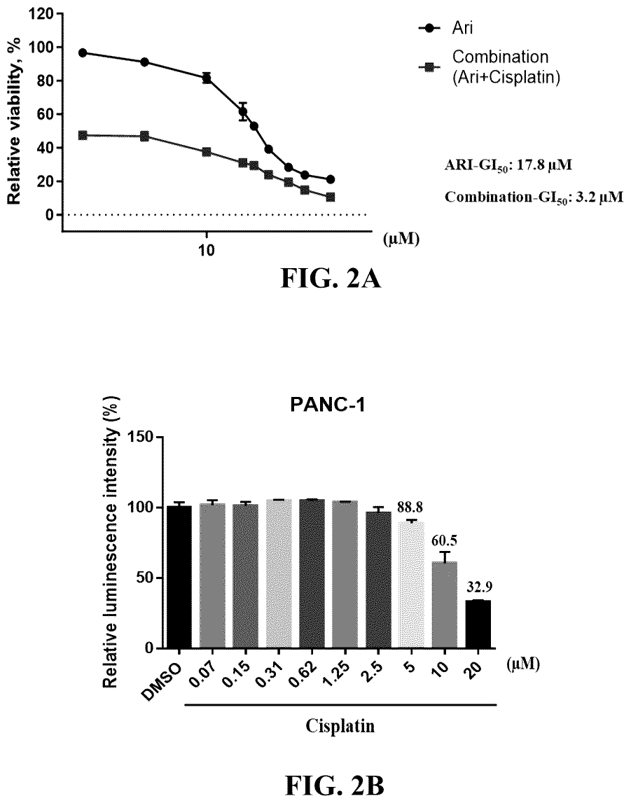

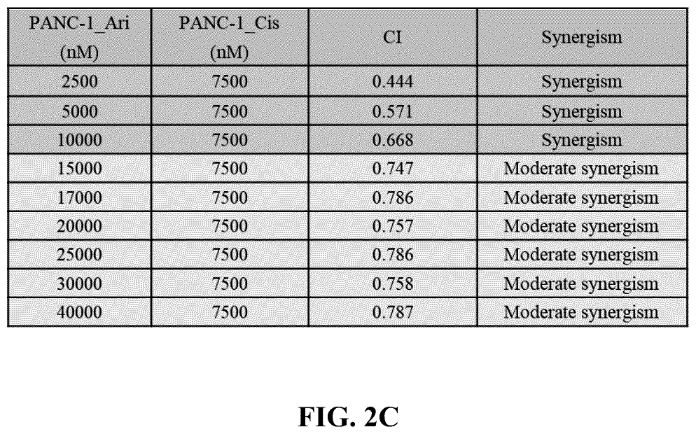

GI₅₀ (or growth inhibition 50, which is the maximum concentration at which the cell proliferation is halved when the drug is administered) for aripiprazole was determined to be 17.8 μM, as shown in FIG. 2A. GI₅₀ for aripiprazole was determined to establish the range i.e., from 2.5 μM to 40 μM for the concentration of aripiprazole to be tested, as shown in FIG. 2C. GI 50 for the combination of aripiprazole and cisplatin was determined to be 3.2 μM, as shown in FIG. 2A. For cisplatin, a concentration at which 75˜85% cell viability was determined to be 7.5 μM (=7500 nM), as shown in FIG. 2B.

As shown in FIG. 2C, at 7.5 μM of cisplatin and between 2.5 μM (=2500 nM) and 10 μM (=10000 nM) of aripiprazole, synergism (CI between 0.3 and 0.7; see FIG. 1A) was observed. At 7.5 μM of cisplatin and between 15 μM (=15000 nM) and 40 μM (=40000 nM) of aripiprazole, moderate synergism (CI between 0.7 and 0.85; see FIG. 1A) was observed. Throughout the entire range of concentration of aripiprazole, the combination of aripiprazole and cisplatin showed higher efficacy in treating pancreatic cancer than the sum of their individual efficacy.

### Example 2

To measure the efficacy of the treatment of pancreatic cancer using aripiprazole and an anti-cancer agent, HPAC, pancreatic human cancer cell line, was cultured and tested.

HPAC cell line was cultured in a 5% CO₂ incubator at 37° C., using Dulbecco's Modified Eagle's Medium (DMEM; Gibco), Roswell Park Memorial Institute (RPMI-1640; Gibco), and Dulbecco's Modified Eagle Medium/Nutrient Mixture F-12 (DMEM-F12; Gibco) supplemented with 10% Fetal Bovine Serum (FBS; Gibco, Rockville MD, USA) and 1% Antibiotic/Antimycotic solution.

For measuring cell growth inhibition, 3,000 HPAC cells per well were incubated overnight. The next day, after chemically treating the cells, the cells were cultured for 3 days to obtain the results. After processing CellTiter-Glo Luminescent cell viability assay (Promega), the value of luminescent was obtained using the multiplate reader (Biotek).

To measure the level of synergism, cell growth was quantified as a percentage through the value obtained through CellTiter-Glo Luminescent cell viability assay, which was converted back into effect values.

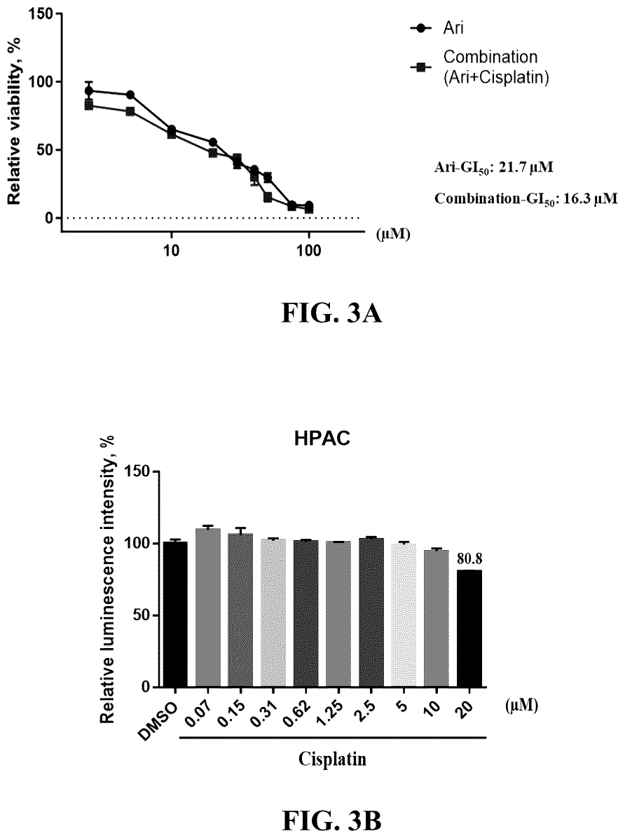

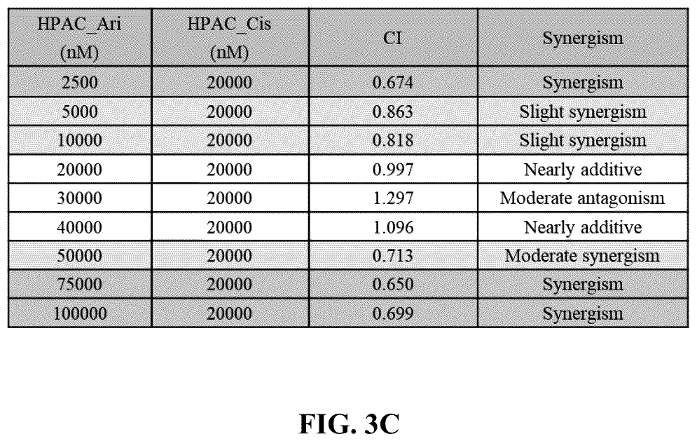

GI₅₀ (or growth inhibition 50, which is the maximum concentration at which the cell proliferation is halved when the drug is administered) for aripiprazole was determined to be 21.7 μM, as shown in FIG. 3A. GI₅₀ for aripiprazole was determined to establish the range i.e., from 2.5 μM to 100 μM for the concentration of aripiprazole to be tested, as shown in FIG. 3C. GI 50 for the combination of aripiprazole and cisplatin was determined to be 16.3 μM, as shown in FIG. 3A.

For cisplatin, a concentration at which 75˜85% cell viability was determined to be 20 μM, as shown in FIG. 3B.

As shown in FIG. 3C, at 20 μM of cisplatin and at 2.5 μM (=2500 nM) of aripiprazole, synergism (CI between 0.3 and 0.7; see FIG. 1A) was observed. At 20 μM of cisplatin and between 75 μM (=75000 nM) and 100 μM (=100000 nM) of aripiprazole, synergism (CI between 0.3 and 0.7; see FIG. 1A) was observed as well. At 20 μM of cisplatin and between 5 μM (=5000 nM) and 10 μM (=10000 nM) of aripiprazole, slight synergism (CI between 0.85 and 0.90; see FIG. 1A) was observed.

Throughout the nearly entire range of concentration of aripiprazole, the combination of aripiprazole and cisplatin showed higher efficacy in treating pancreatic cancer than the sum of their individual efficacy.

### Example 3

To measure the efficacy of the treatment of glioma using aripiprazole and an anti-cancer agent, U-251 MG, human glioma cancer cell line, was cultured and tested.

U-251 MG cell line was cultured in a 5% CO₂ incubator at 37° C., using Dulbecco's Modified Eagle's Medium (DMEM; Gibco), Roswell Park Memorial Institute (RPMI-1640; Gibco), and Dulbecco's Modified Eagle Medium/Nutrient Mixture F-12 (DMEM-F12; Gibco) supplemented with 10% Fetal Bovine Serum (FBS; Gibco, Rockville MD, USA) and 1% Antibiotic/Antimycotic solution.

For measuring cell growth inhibition, 3,000 U-251 MG cells per well were incubated overnight. The next day, after chemically treating the cells, the cells were cultured for 3 days to obtain the results. After processing CellTiter-Glo Luminescent cell viability assay (Promega), the value of luminescent was obtained using the multiplate reader (Biotek).

To measure the level of synergism, cell growth was quantified as a percentage through the value obtained through CellTiter-Glo Luminescent cell viability assay, which was converted back into effect values.

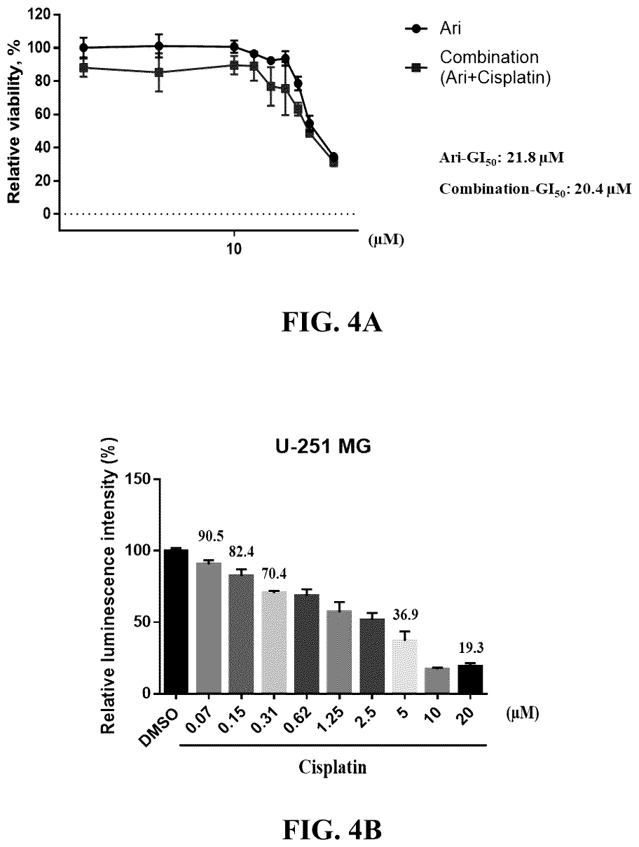

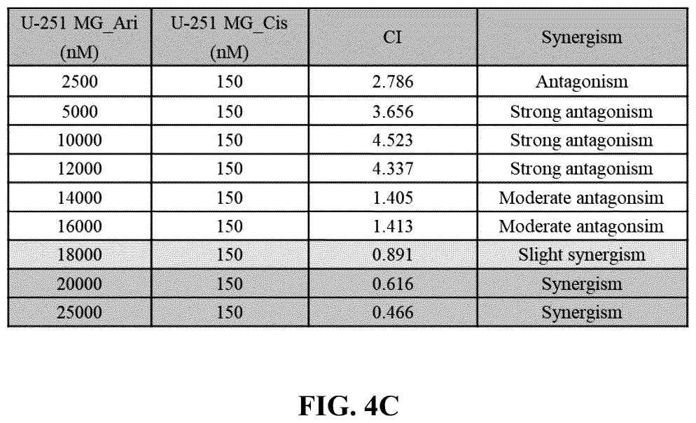

GI₅₀ (or growth inhibition 50, which is the maximum concentration at which the cell proliferation is halved when the drug is administered) for aripiprazole was determined to be 21.8 μM, as shown in FIG. 4A. GI₅₀ for aripiprazole was determined to establish the range i.e., from 2.5 μM to 25 μM for the concentration of aripiprazole to be tested, as shown in FIG. 4C. GI 50 for the combination of aripiprazole and cisplatin was determined to be 20.4 μM, as shown in FIG. 4A.

For cisplatin, a concentration at which 75˜85% cell viability was determined to be 0.15 μM (=150 nM), as shown in FIG. 4B.

As shown in FIG. 4C, at 0.15 μM of cisplatin and between 20 μM (=20000 nM) and 25 μM (=25000 nM) of aripiprazole, synergism (CI between 0.3 and 0.7; see FIG. 1A) was observed. At 0.15 μM of cisplatin and at 18 μM (=18000 nM) of aripiprazole, slight synergism (CI between 0.85 and 0.90; see FIG. 1A) was observed.

Throughout the range of concentration of aripiprazole (>18 μM), the combination of aripiprazole and cisplatin showed higher efficacy in treating glioma than the sum of their individual efficacy.

### Example 4

To measure the efficacy of the treatment of glioma using aripiprazole and an anti-cancer agent, U-87 MG, human glioma cancer cell line, was cultured and tested.

U-87 MG cell line was cultured in a 5% CO₂ incubator at 37° C., using Dulbecco's Modified Eagle's Medium (DMEM; Gibco), Roswell Park Memorial Institute (RPMI-1640; Gibco), and Dulbecco's Modified Eagle Medium/Nutrient Mixture F-12 (DMEM-F12; Gibco) supplemented with 10% Fetal Bovine Serum (FBS; Gibco, Rockville MD, USA) and 1% Antibiotic/Antimycotic solution.

For measuring cell growth inhibition, 3,000 U-87 MG cells per well were incubated overnight. The next day, after chemically treating the cells, the cells were cultured for 3 days to obtain the results. After processing CellTiter-Glo Luminescent cell viability assay (Promega), the value of luminescent was obtained using the multiplate reader (Biotek).

To measure the level of synergism, cell growth was quantified as a percentage through the value obtained through CellTiter-Glo Luminescent cell viability assay, which was converted back into effect values.

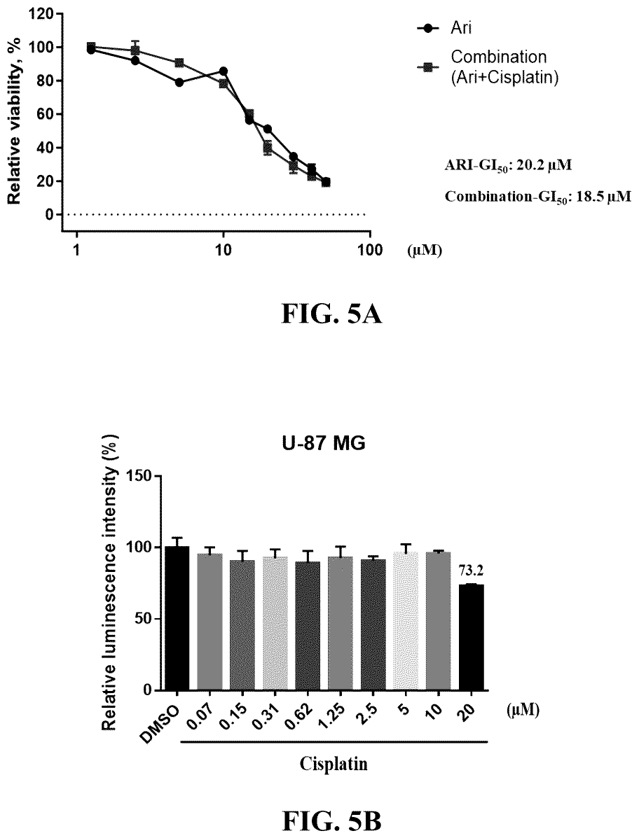

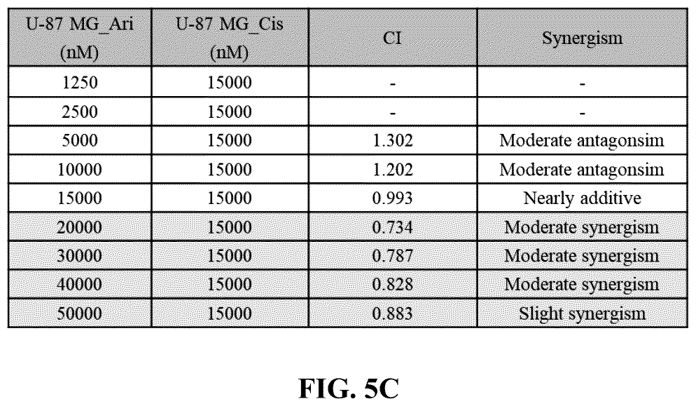

GI₅₀ (or growth inhibition 50, which is the maximum concentration at which the cell proliferation is halved when the drug is administered) for aripiprazole was determined to be 20.2 μM, as shown in FIG. 5A. GI₅₀ for aripiprazole was determined to establish the range i.e., from 1.25 μM to 50 μM for the concentration of aripiprazole to be tested, as shown in FIG. 5C. GI 50 for the combination of aripiprazole and cisplatin was determined to be 18.5 μM (=18500 nM), as shown in FIG. 5A

For cisplatin, a concentration at which 75˜85% cell viability was determined to be 15 μM, as shown in FIG. 5B.

As shown in FIG. 5C, at 15 μM of cisplatin and between 20 μM (=20000 nM) and 40 μM (=40000 nM) of aripiprazole, moderate synergism (CI between 0.7 and 0.85; see FIG. 1A) was observed. At 15 μM of cisplatin and at 50 μM (=50000 nM) of aripiprazole, slight synergism (CI between 0.85 and 0.90; see FIG. 1A) was observed.

Throughout the range of concentration of aripiprazole (>20 μM), the combination of aripiprazole and cisplatin showed higher efficacy in treating glioma than the sum of their individual efficacy.

Examples 1-4 suggest that the very same combination would not be equally effective in treating two different cancers. For some cancers, synergism may be concentration-dependent. Also, even for the same cancer, the very same combination of aripiprazole and anticancer agent may show different efficacy or synergism, depending on the tested cell lines.

### Example 5

To measure the efficacy of the treatment of colorectal cancer using aripiprazole and an anti-cancer agent, HCT-116, human colorectal cancer cell line, was cultured and tested.

HCT-116 cell line was cultured in a 5% CO₂ incubator at 37° C., using Dulbecco's Modified Eagle's Medium (DMEM; Gibco), Roswell Park Memorial Institute (RPMI-1640; Gibco), and Dulbecco's Modified Eagle Medium/Nutrient Mixture F-12 (DMEM-F12; Gibco) supplemented with 10% Fetal Bovine Serum (FBS; Gibco, Rockville MD, USA) and 1% Antibiotic/Antimycotic solution.

For measuring cell growth inhibition, 3,000 HCT-116 cells per well were incubated overnight. The next day, after chemically treating the cells, the cells were cultured for 3 days to obtain the results. After processing CellTiter-Glo Luminescent cell viability assay (Promega), the value of luminescent was obtained using the multiplate reader (Biotek).

To measure the level of synergism, cell growth was quantified as a percentage through the value obtained through CellTiter-Glo Luminescent cell viability assay, which was converted back into effect values.

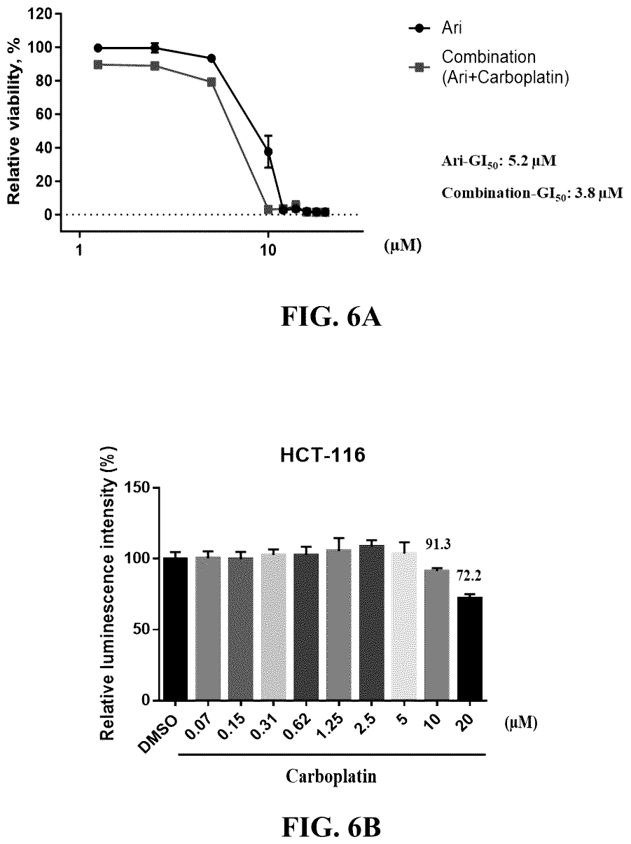

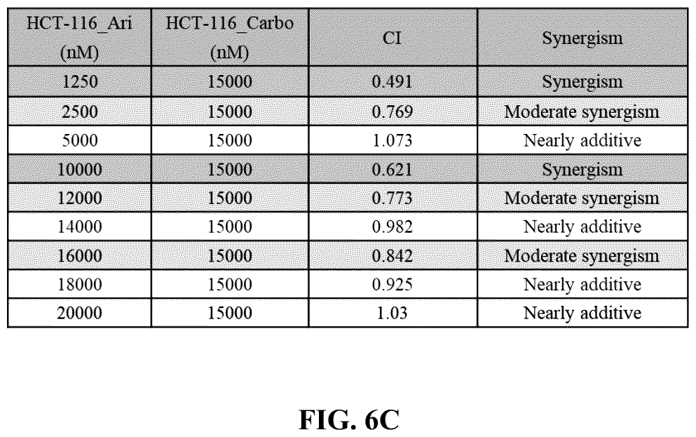

GI₅₀ (or growth inhibition 50, which is the maximum concentration at which the cell proliferation is halved when the drug is administered) for aripiprazole was determined to be 5.2 μM, as shown in FIG. 6A. GI₅₀ for aripiprazole was determined to establish the range i.e., from 1.25 μM to 20 μM for the concentration of aripiprazole to be tested, as shown in FIG. 6C. GI 50 for the combination of aripiprazole and cisplatin was determined to be 3.8 μM, as shown in FIG. 6A.

For cisplatin, a concentration at which 75˜85% cell viability was determined to be 15 μM (=15000 nM), as shown in FIG. 6B.

As shown in FIG. 6C, at 15 μM of cisplatin and at 1.25 μM (=1250 nM) and 10 μM (=10000 nM) of aripiprazole, synergism (CI between 0.3 and 0.7; see FIG. 1A) was observed. At 15 μM of cisplatin and at 2.5 μM (=2500 nM), 12 μM (=12000 nM) and 16 μM (=16000 nM) of aripiprazole, moderate synergism (CI between 0.7 and 0.85; see FIG. 1A) was observed.

Throughout the particular concentrations of aripiprazole, the combination of aripiprazole and cisplatin showed higher efficacy in treating colorectal cancer than the sum of their individual efficacy.

### Comparative Example 1

To measure the efficacy of the treatment of pancreatic cancer using aripiprazole and an anti-cancer agent, PANC-1, pancreatic human cancer cell line, was cultured and tested.

PANC-1 cell line was cultured in a 5% CO₂ incubator at 37° C., using Dulbecco's Modified Eagle's Medium (DMEM; Gibco), Roswell Park Memorial Institute (RPMI-1640; Gibco), and Dulbecco's Modified Eagle Medium/Nutrient Mixture F-12 (DMEM-F12; Gibco) supplemented with 10% Fetal Bovine Serum (FBS; Gibco, Rockville MD, USA) and 1% Antibiotic/Antimycotic solution.

For measuring cell growth inhibition, 3,000 PANC-1 cells per well were incubated overnight. The next day, after chemically treating the cells, the cells were cultured for 3 days to obtain the results. After processing CellTiter-Glo Luminescent cell viability assay (Promega), the value of luminescent was obtained using the multiplate reader (Biotek).

To measure the level of synergism, cell growth was quantified as a percentage through the value obtained through CellTiter-Glo Luminescent cell viability assay, which was converted back into effect values.

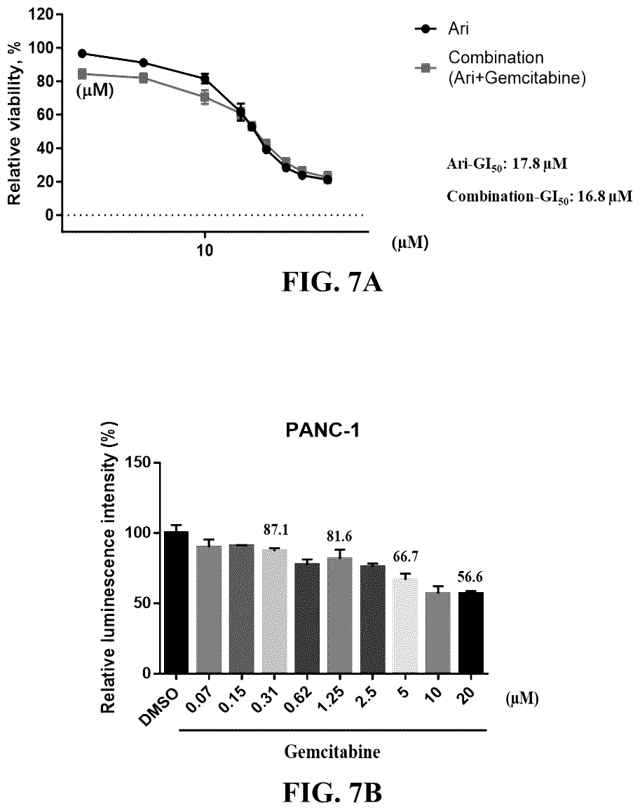

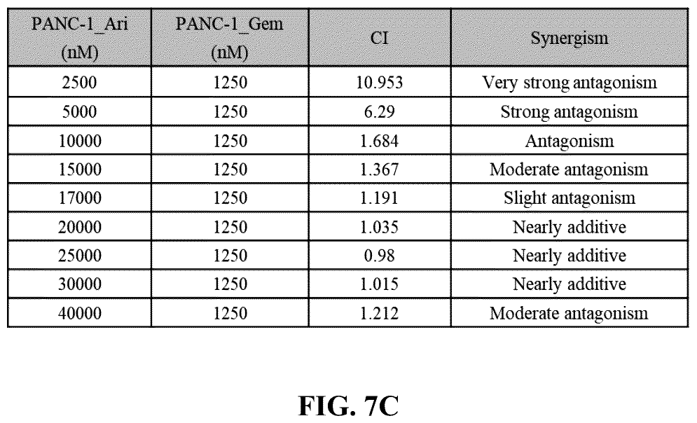

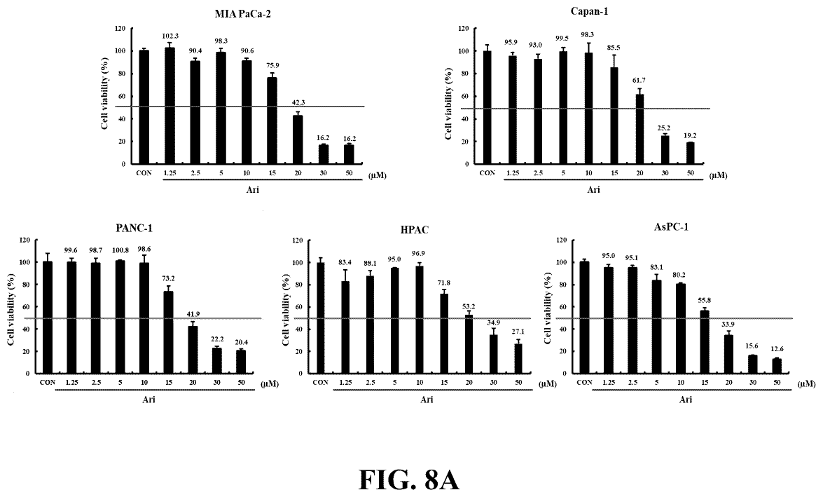

GI₅₀ (or growth inhibition 50, which is the maximum concentration at which the cell proliferation is halved when the drug is administered) for aripiprazole was determined to be 17.8 μM, as shown in FIG. 7A. GI₅₀ for aripiprazole was determined to establish the range i.e., from 2.5 μM to 40 μM for the concentration of aripiprazole to be tested, as shown in FIG. 7C. GI 50 for the combination of aripiprazole and gemcitabine was determined to be 16.8 μM, as shown in FIG. 7A.

For gemcitabine, a concentration at which 75˜85% cell viability was determined to be 1.25 μM, as shown in FIG. 7B.

As shown in FIG. 7C, at 1.25 μM of gemcitabine and between 2.5 μM (=2500 nM) and 40 μM (=40000 nM) of aripiprazole, no synergism was observed. At 1.25 μM of gemcitabine and at 2.5 μM (=2500 nM) of aripiprazole, very strong antagonism (CI >10; see FIG. 1A) was observed. At 1.25 μM of gemcitabine and at 5.0 μM (=5000 nM) of aripiprazole, strong antagonism (CI between 3.3 and 10; see FIG. 1A) was observed. At 1.25 μM of gemcitabine and at 10.0 μM (=10000 nM) of aripiprazole, antagonism (CI between 1.45 and 3.3; see FIG. 1A) was observed. At 1.25 μM of gemcitabine and at 15.0 μM (=15000 nM) and 40.0 μM (=40000 nM) of aripiprazole, moderate antagonism (CI between 1.20 and 1.45; see FIG. 1A) was observed. At 1.25 μM of gemcitabine and at 17.0 μM (=17000 nM) of aripiprazole, slight antagonism (CI between 1.10and 1.20; see FIG. 1A) was observed. At 1.25 μM of gemcitabine and between 20.0 μM (=20000 nM) and 30.0 μM (=30000 nM) of aripiprazole, nearly additive (CI between 0.90 and 1.10; see FIG. 1A) was observed.

This Comparative Example 1 suggests that not every combination of aripiprazole and an anticancer agent result in synergism in treating cancer. The present invention surprisingly discovered which combination would result in high level of synergism in treating cancer. It is, however, noted that the efficacy of aripiprazole, anticancer agent or both in treating cancer cell lines may depend on cancer cell lines. It is possible that some cancer cell lines are more resistant to aripiprazole, anticancer agent or both. In other words, the efficacy or synergism of aripiprazole, anticancer agent or both may depend on cancer cell lines.

### Mechanism Behind the Synergistic Cancer Treatment—Aripiprazole'S Chemo-Sensitizing Role via STAT3 Inhibition and Pro-Apoptotic Pathway Activation

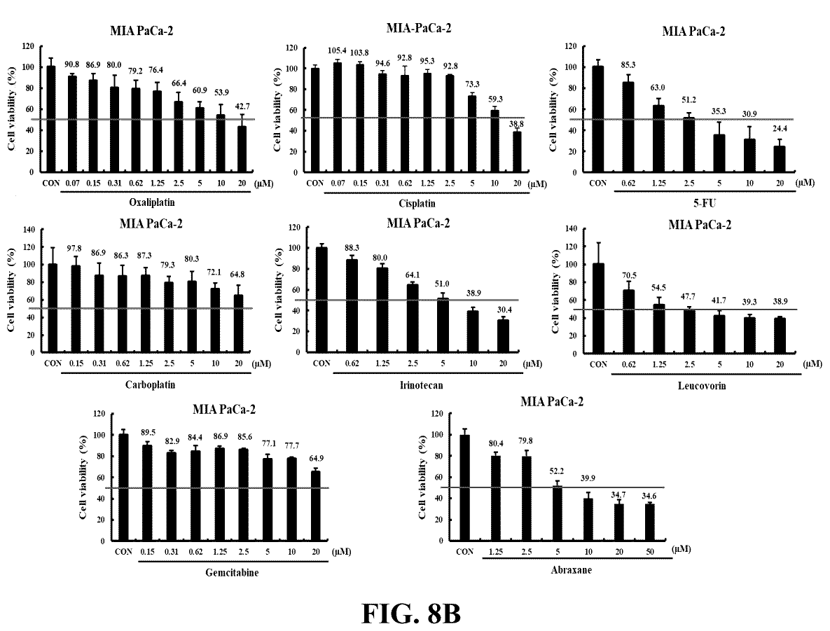

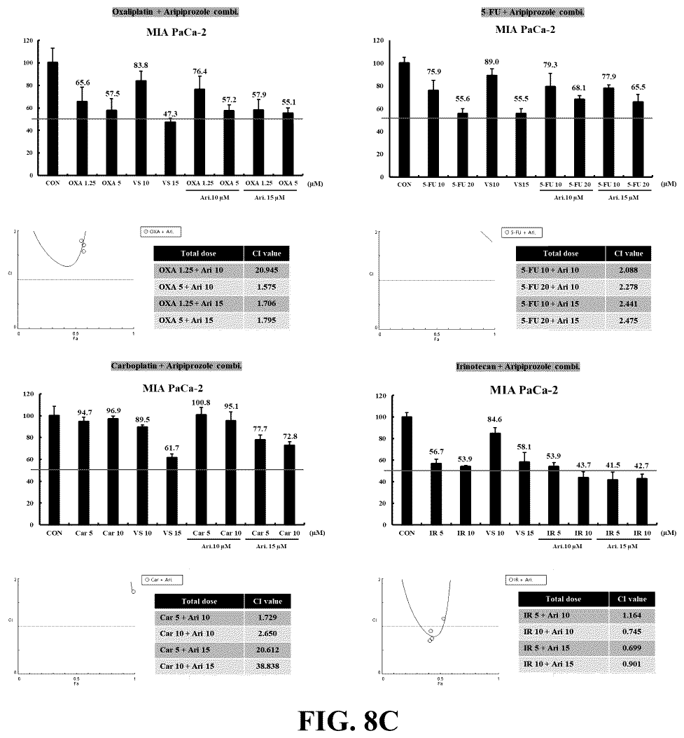

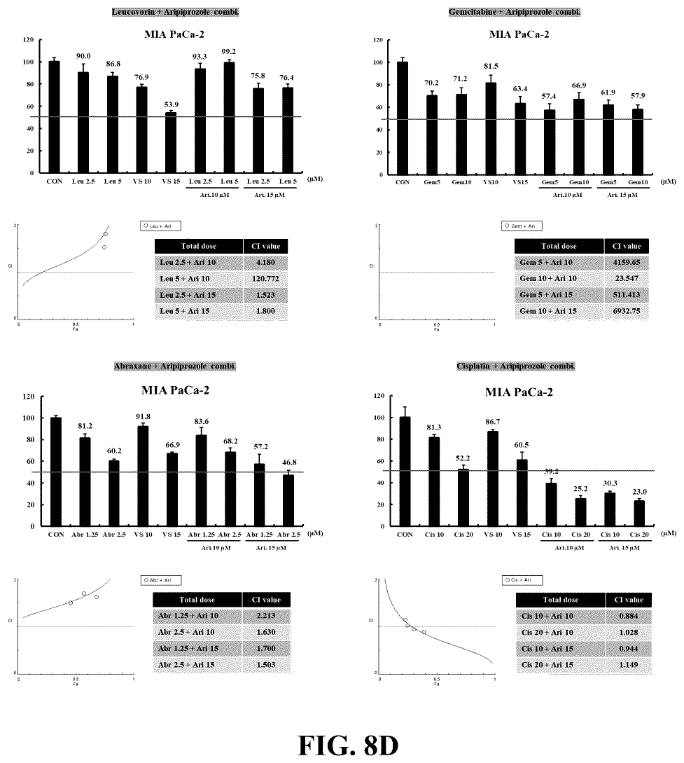

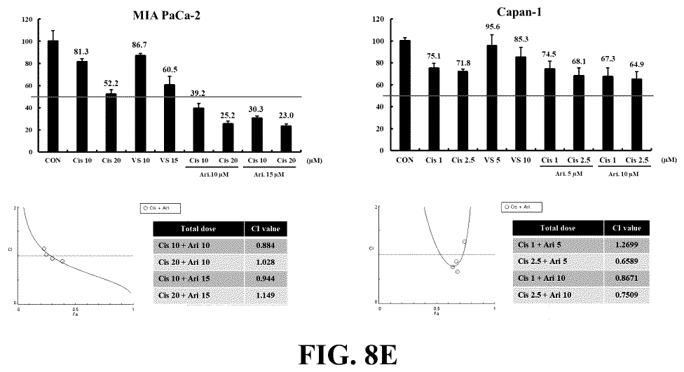

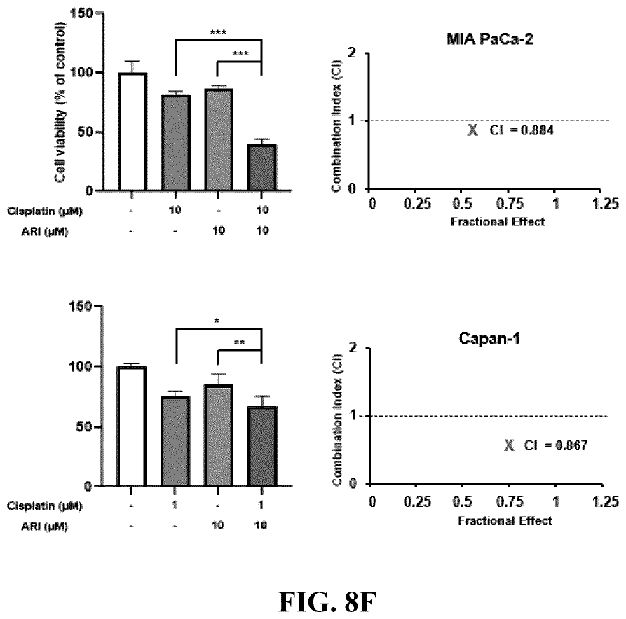

The effect of anticancer drugs on cell viability was identified in five pancreatic cancer cell lines (MIA PaCa-2, Capan-1, PANC-1, HPAC, AsPC-1). MTT assay was used to test cell viability of the pancreatic cancer cell lines, as shown in FIG. 8A. Five pancreatic cancer cell lines (MIA PaCa-2, PANC-1, AsPC-1, HPAC, Capan-1) were seeded in 96-well plates at 2500, 3000, 5000 cells per well, respectively. After 24 hours, each cell line was treated with different concentrations of aripiprazole and cisplatin, serially diluted to 0.07-50 μM in culture medium. After 48 hours, 2 mg/mL MTT solution was added at a ratio of 1/10 of the culture medium and incubated in a 5% CO₂ incubator at 37° C. for 4 hours. Then, 200 μL of DMSO was added and absorbance was measured at 540 nm. Aripiprazole inhibited the growth of pancreatic cancer cells at a concentration of 10 μM, and more than 70% of cells began to die from 30 μM.

Among the five pancreatic cancer cell lines, MIA PaCa-2 and Capan-1 were selected because they showed a stable and significant combination effects.

Among the anticancer drugs in clinical use, cisplatin, which showed the most synergistic results among a total of eight anticancer drugs in MIA PaCa-2 cells, was selected to identify anticancer efficacy and mechanisms, as shown in FIGS. 8B-8D.

MTT assay was conducted for both MIA PaCa-2 and Capan-1, as shown in FIG. 8E. MIA PaCa-2 cells were treated with aripiprazole (10 μM and 15 μM), cisplatin (10 μM and 20 μM) and their combination for 48 hours, and Capan-1 cells were treated with aripiprazole (5 μM and 10 μM), cisplatin (1 μM and 2.5 μM) and their combination for 48 hours. Compared to treatment with either agent alone, co-treatment with both agents caused greater inhibition of cell growth. To identify synergistic effects of aripiprazole and cisplatin, combination index (CI) values were calculated using CompuSyn v1.0 (Biosoft). Aripiprazole and cisplatin produced significant synergistic effects, with CI values<1 for the combination of 10 μM aripiprazole and 10 μM cisplatin in MIA PaCa-2 cells (CI=0.884) and 10 μM aripiprazole and 1 μM cisplatin in Capan-1 (CI=0.867) cells. As shown in FIG. 8F, the cytotoxicity of aripiprazole and cisplatin in MIA PaCa-2 and Capan-1 cells was evaluated using an MTT assay over 48 hours of treatment with cisplatin (1 μM or 10 μM), aripiprazole (10 μM), or a combination of both. Each measured value is presented as the mean±standard deviation (n=4). For both MIA PaCa-2 and Capan-1, the combination index (CI) fell below 1, indicating a statistically meaningful synergistic interaction between cisplatin and aripiprazole.

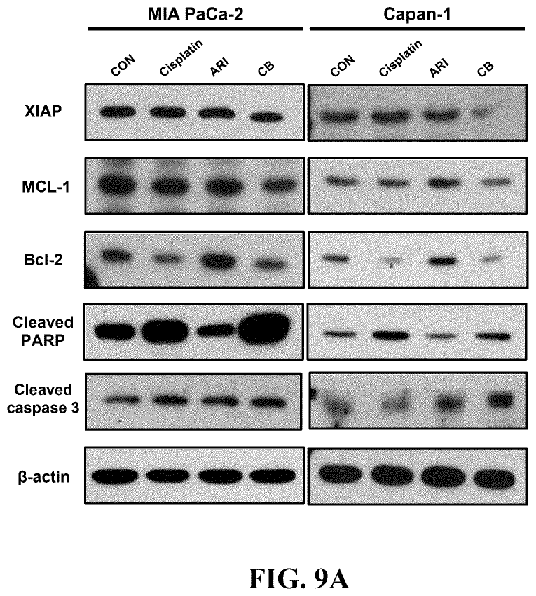

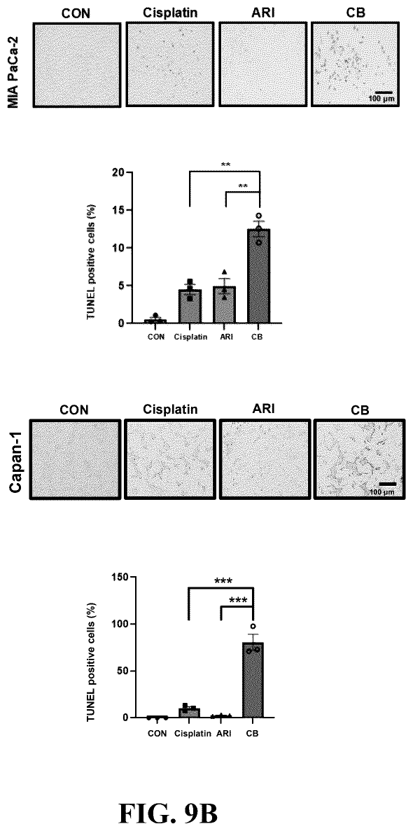

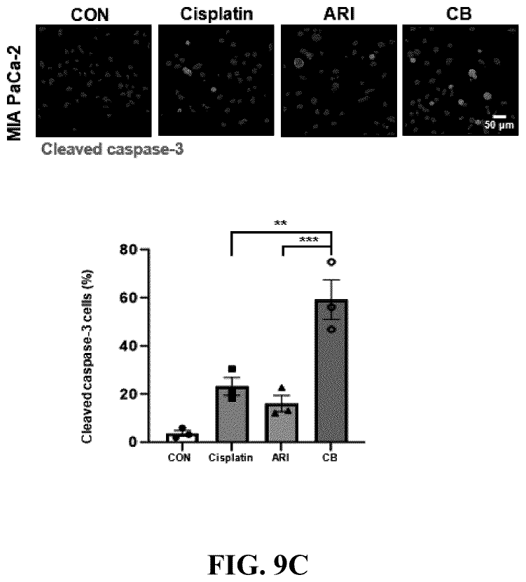

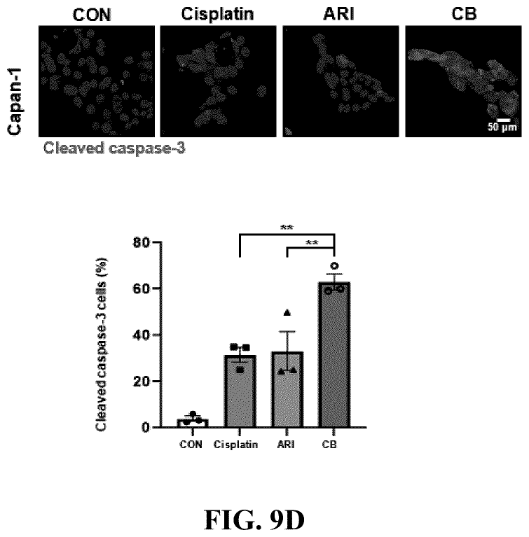

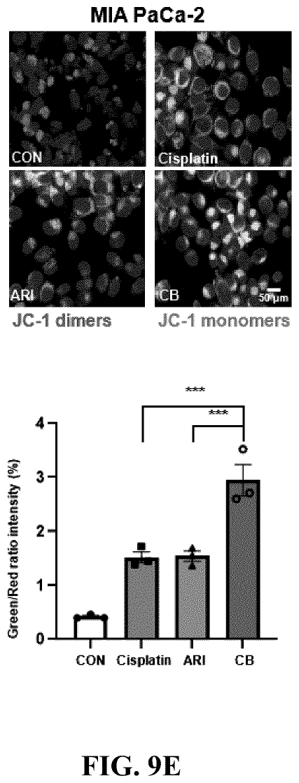

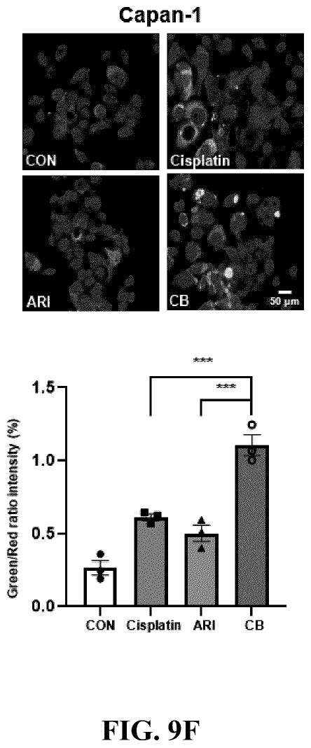

In determining the mechanism of action in treating pancreatic cancer using aripiprazole and cisplatin, FIG. 9A provides a comprehensive Western blot analysis of apoptosis-related markers in MIA PaCa-2 and Capan-1 cells. For the Western blotting, 1×10\hat{ }6 MIA PaCa-2 or Capan-1 cells were seeded in 100 mm dishes. After 24 hours, the cells were divided into four groups and treated with: (i) medium only (Control), (ii) cisplatin (Cisplatin Group), (iii) aripiprazole (Aripiprazole Group), or (iv) a combination of cisplatin and aripiprazole (COMBI). The cells were then incubated at 37° C. in a 5% CO₂ incubator for 48 hours. Lysis was performed at 4° C. for 30 minutes using RIPA buffer containing protease and phosphatase inhibitors, followed by centrifugation at 13,000 rpm for 30 minutes to collect the protein supernatant. Each sample (50 μg protein) was separated on a 12% SDS-PAGE gel, transferred to a PVDF membrane, and blocked with 5% non-fat dry milk. Membranes were then incubated overnight at 4° C. with primary antibodies against Cleaved PARP (1:1000, CST #9541), PARP (1:1000, CST #9532), MCL-1(1:1000, SC-819), Bcl-2 (1:1000, CST #2876), and β-actin (1:10000, CST #4967). An anti-rabbit secondary antibody (1:2000, CST #7076S) was applied for 1 hour, followed by ECL detection.

As depicted in FIG. 9A, both MIA PaCa-2 (treated with 10 μM cisplatin and 10 μM aripiprazole) and Capan-1 (treated with 1 μM cisplatin and 10 μM aripiprazole) showed the greatest increase in Cleaved PARP within the combination group (COMBI), alongside the most pronounced decrease in Bcl-2 and MCL-1 expression. Furthermore, the dual-treatment condition exhibited the highest level of Cleaved Caspase-3, indicating heightened cell death through intrinsic apoptotic pathways. Consistent with this, the expression of key anti-apoptotic proteins such as XIAP and MCL-1 was markedly downregulated in the combination group, reinforcing the notion that this regimen suppresses critical survival signals in pancreatic cancer cells. Collectively, these findings confirm that combining aripiprazole with cisplatin enhances pro-apoptotic signaling to a greater extent than either agent alone, thereby promoting increased cell death in MIA PaCa-2 and Capan-1 pancreatic cancer cells.

Additional TUNEL assay (FIG. 9B), cleaved caspase-3 staining using Immuno-cytochemistry (ICC) (FIGS. 9C-9D), and JC-1 staining (FIG. 9E) all confirmed that the Combination group (COMBI; Cisplatin+Aripiprazole) resulted in more cell deaths or apoptosis than in the control and aripiprazole and cisplatin alone groups.

FIG. 9B shows the results of TUNEL assays performed on MIA PaCa-2 and Capan-1 pancreatic cancer cells after 48 hours of treatment. MIA PaCa-2 cells were seeded at 4×10\hat{ }4 cells/well in 12-well plates with 18 mm glass coverslips, while Capan-1 cells were seeded at 2×10\hat{ }5 cells/well under the same conditions. Each cell line was divided into four groups: (i) Control (medium only), (ii) Cisplatin, (iii) Aripiprazole, and (iv) Combination (COMBI; Cisplatin+Aripiprazole). After 48 hours of incubation at 37° C. in a 5% CO₂ atmosphere, cells were fixed with 4% PFA and stored at −20° C. The TUNEL assay was then conducted using the ApopTag Peroxidase In Situ Apoptosis Detection Kit (Merck Millipore), following the manufacturer's protocol. Briefly, permeabilization was performed with 0.5% Triton X-100 for 10 minutes, followed by treatment with equilibration buffer for 1 minute. The TdT enzyme/reaction buffer mixture was applied and incubated for 2 hours at 37° C. The Stop/Wash buffer was added, and anti-digoxigenin peroxidase was applied overnight at 4° C. After three washes in DPBS, DAB substrate was applied for 7 minutes, and the samples were then mounted (Fluorescent Mounting Medium, Vector Laboratories) and photographed under a microscope.

In this assay, MIA PaCa-2 cells received 10 μM of cisplatin, 10 μM of aripiprazole, or their combination, whereas Capan-1 cells were treated with 1 μM of cisplatin, 10 μM of aripiprazole, or the combination. As illustrated in FIG. 9B, a notably higher fraction of TUNEL-positive (apoptotic) cells was observed in the combination (COMBI) group compared to either cisplatin alone or aripiprazole alone. This finding underscores the enhanced capacity of cisplatin+aripiprazole to induce programmed cell death (apoptosis) in both pancreatic cancer cell lines, thereby suggesting a synergistic effect of these agents on the apoptotic pathway.

FIGS. 9C and 9D depict immunocytochemical detection of cleaved caspase-3 in MIA PaCa-2 and Capan-1 cells, respectively. MIA PaCa-2 cells (4×10\hat{ }4 cells/well) and Capan-1 cells (2×10\hat{ }5 cells/well) were seeded onto 18 mm glass coverslips in 12-well plates and divided into four groups: Control (medium only), Cisplatin, Aripiprazole, and Combination (COMBI; Cisplatin+Aripiprazole). After 48 hours, the cells were fixed with 4% PFA, permeabilized with 0.5% Triton X-100, and blocked for 1 hour at room temperature. A primary antibody against cleaved caspase-3 (1:30, CST #9661S) was applied overnight at 4° C., followed by a fluorescently tagged secondary antibody (e.g., FITC or a red fluorophore). DAPI (1:100) was used for nuclear staining before coverslips were mounted and imaged via confocal microscopy. MIA PaCa-2 cells received 10 μM cisplatin, 10 μM aripiprazole, or the combination, while Capan-1 cells were treated with 1 μM cisplatin, 10 μM aripiprazole, or the combination. In both cell lines, the dual therapy (COMBI) consistently exhibited stronger cleaved caspase-3 staining, signifying a more robust apoptotic response compared to either monotherapy or the untreated control.

For MIA PaCa-2, 10 μM cisplatin, 10 μM aripiprazole, or the combination (COMBI) was applied for 48 hours. For Capan-1, 1 μM cisplatin, 10 μM aripiprazole, or the combination was used under the same incubation conditions. FIGS. 9C and 9D display immunofluorescent detection of cleaved caspase-3, wherein the combination therapy consistently exhibits stronger cleaved caspase-3 (green) staining-indicative of a more robust apoptotic response-compared to either monotherapy or the untreated control.

In both MIA PaCa-2 and Capan-1 cells, the enhanced cleaved caspase-3 staining in the COMBI group supports the notion that aripiprazole augments cisplatin-mediated cell death pathways. These data align with other assays indicating that dual treatment triggers a more pronounced pro-apoptotic effect than either cisplatin or aripiprazole alone, thus underscoring the therapeutic potential of combining dopaminergic modulation with standard chemotherapy in pancreatic cancer cells.

FIG. 9E and FIG. 9F illustrate the results of JC-1 staining in MIA PaCa-2 and Capan-1 cells, respectively. MIA PaCa-2 cells (4×10\hat{ }4 cells/well) and Capan-1 cells (2×10\hat{ }5 cells/well) were seeded onto 18 mm glass coverslips in 12-well plates and assigned to four treatment groups: Control (no drug), Cisplatin, Aripiprazole, and Combination (COMBI; Cisplatin+Aripiprazole). After 48 hours at 37° C. in a 5% CO₂ incubator, JC-1 reagent (No. 10009908) was added at a 1:100 ratio in culture medium and incubated for 45 minutes under the same conditions. The cells were then fixed with 4% PFA for 5 minutes, washed three times, stained with DAPI (1:100) for 30 minutes, and mounted using fluorescent mounting medium (Vector Laboratories) prior to confocal microscopy.

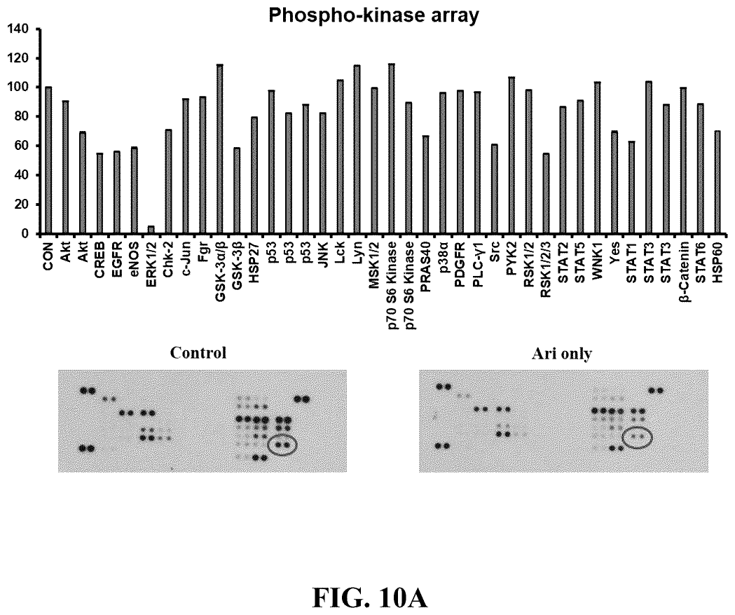

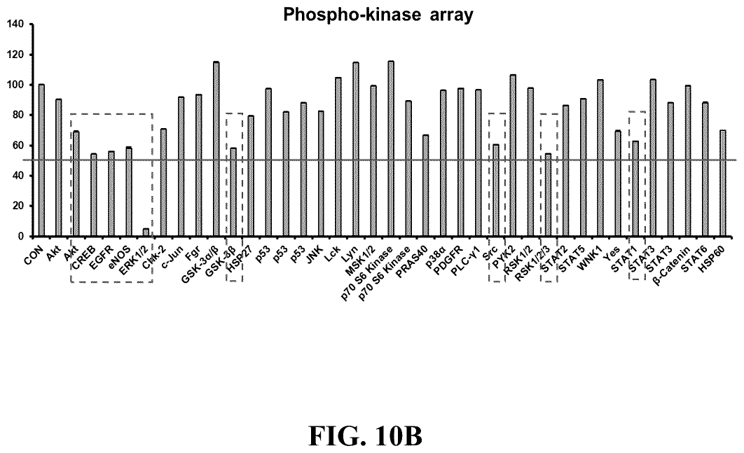

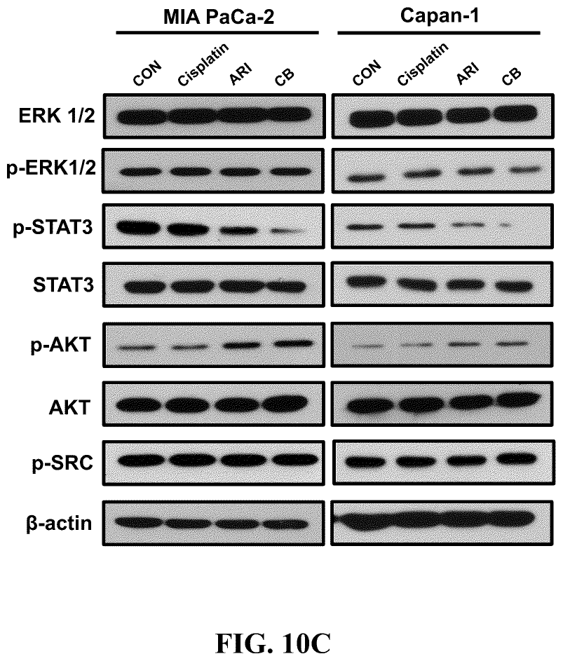

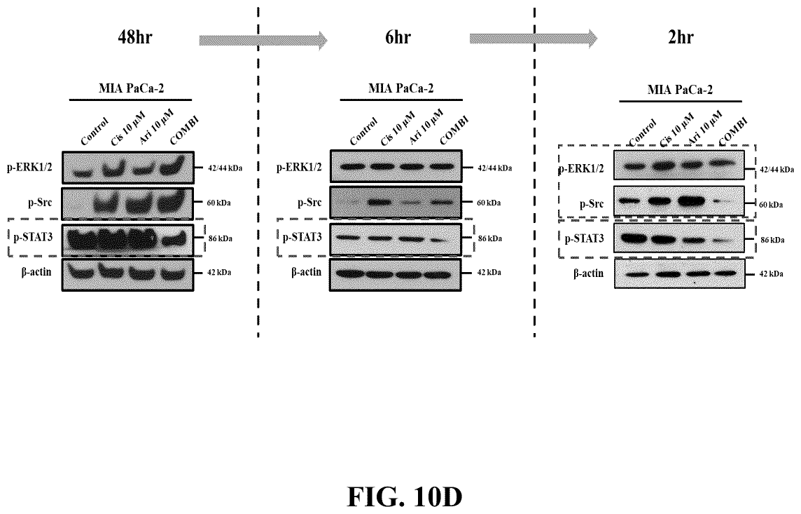

For MIA PaCa-2 cells, 10 μM cisplatin, 10 μM aripiprazole, or their combination was used, while Capan-1 cells received 1 μM cisplatin, 10 μM aripiprazole, or the combination, all for 48 hours. In healthy mitochondria with high membrane potential (MMP), JC-1 accumulates in aggregates and emits red fluorescence; when apoptosis occurs and the MMP collapses, JC-1 remains in monomeric form, emitting green fluorescence in the cytosol. In both MIA PaCa-2 and Capan-1, the COMBI group displayed a more pronounced shift from red to green compared to either single-agent treatment or the control, indicating increased mitochondrial membrane depolarization and thus heightened apoptosis. These findings underscore a synergistic effect of co-treating with cisplatin and aripiprazole in disrupting mitochondrial integrity and promoting apoptotic cell death in pancreatic cancer cells. As shown in FIG. 10A, to discover a new mechanism of action in treating pancreatic cancer using aripiprazole and cisplatin, Phospho-kinase array in MIA PaCa-2 cell was prepared and tested. The array used chips implanted with 42 phospho-kinases mediating cell signaling known as anticancer mechanisms, which identified key signaling involving the combination of aripiprazole and cisplatin.

For phospho-kinase array testing, MIA PaCa-2 cells were seeded in 100 nm-dish with 1×10⁶ cells, and after 24 hours, the drug is treated in two groups, Media Group (CON) and Aripiprazole group, and then incubated in a 5% CO₂ incubator at 37° C. for 2 and 6 hours, respectively.

Human phosphor-Kinase Array Kit (R&D systems, No. ARY003C) was used. Blocking with Array buffer 1 for 1 hour for each member in each kit, then adjust the prepared protein to 600 ug and mix with Array buffer 2. After that, spray it on the blocking-finished membrane and attach it at 4° C. for overnight. The next day, after going through the wash process 3 times with 1× wash buffer, attach antibodies corresponding to 1× Array buffer ⅔ respectively, and shake it on RT for 2 hours. After that, after performing the wash process once more, CHEMI REAGENT 1 and 2 were mixed, sprayed on the membrane, and detected.

As shown in FIG. 10B, the test confirmed that the STAT groups, ERK, and Src pathway reduced the most. As shown in FIG. 10C, for the newly discovered signaling, Western blotting (6 hours) was conducted to confirm the results of phosphor-kinase array using control, cisplatin, aripiprazole, and combination thereof. For the Western blotting, MIA PaCa-2, Capan-1 cells were seeded in 100 nm-dish at 1×10⁶ cells. After 24 hours, the cells were divided into four groups and treated with each drug: Media group (Control), Cisplatin Group, Aripiprazole Group, and Combination Group (COMBI; Cisplatin+Aripiprazole), and then incubated in a 5% CO₂ incubator at 37° C. for 48 hours.

Lysis was performed at 4° C. for 30 minutes using RIPA buffer containing 1× protease inhibitor and 1× phosphatase inhibitor and then centrifuged at 13,000 RPM for 30 minutes to extract protein. 50 μg of protein was separated using a 12% SDS-PAGE gel and the protein was transferred to the Polyvinylidene fluoride (PVDF) membrane. Membrane was blocked with 5% non-fat skin milk and then reacted overnight at 4° C. with a primary antibody diluted in PBS-T including 5% BSA. For the primary antibody, ERK1/2 (1:1000, 9102), p-ERK1/2 (1:1000, 4377), STAT3 (1:1000, 9139), p-STAT3 (1:1000, 94994), AKT (1:1,000, 9272), p-AKT (1:2,000, 4060), p-Src (1:1000, 6943), β-actin (1:10000, 4967) purchased from Cell signaling Technology (Beverly, MA, USA) were used. Anti-rabbit (1:2000, 7076S) purchased from Cell signaling Technology (Beverly, MA, USA) was used as a secondary antibody, shaken for 1 hour, and detection was performed using ECL.

The expressions of p-ERK in each were the same, while p-Src showed different tendencies in MIA PaCa-2 and Capan-1. In both MIA PaCa-2 and Capan-1, p-STAT3 expression reduced the most.

FIG. 10D illustrates the Western blotting results for 48 hours, 6 hours, and 2 hours drug treatment of MIA PaCa-2 with control, cisplatin, aripiprazole, and combination of cisplatin and aripiprazole. The number of MIA PaCa-2 cells was 1×10⁶ (100 pi/1 plate).

As shown in FIG. 10D, with the 48 hours treatment, p-ERK1/2 increased in cisplatin and combination; with the 6 hours treatment, no change; and with the 2 hours treatment, slightly decreased in combination due to aripiprazole. No consistent trend was observed for the marker p-ERK1/2. With the 48 hours treatment, p-Src increased in aripiprazole and combination; with the 6 hours treatment, increased in cisplatin; and with the 2 hours treatment, increased in aripiprazole but decreased in combination. No consistent trend was observed for the marker p-Src. With all the 48 hours, 6 hours, and 2 hours treatment, p-STAT3 was observed to decrease in combination.

The results suggest that STAT3 is involved the most in the synergistic pancreatic cancer treatment with the combination of aripiprazole and cisplatin. Based on the results of this study, if the effect is confirmed in vivo experiments through xenograft animal model using pancreatic cancer cell lines, it is expected that it can be applied to clinical practice in the future.

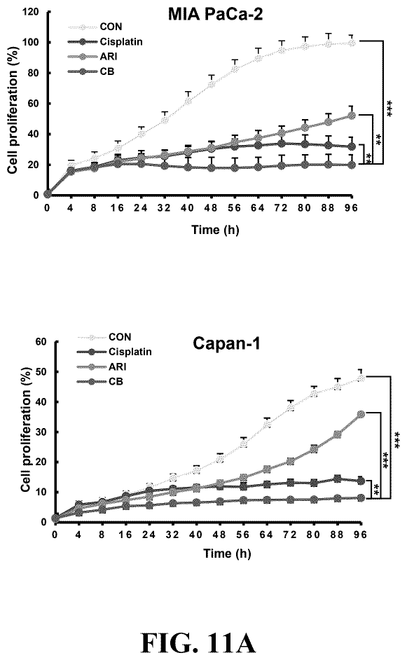

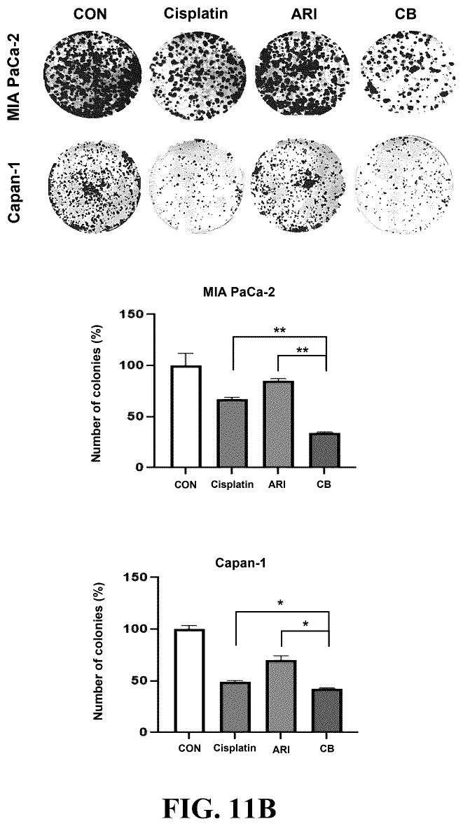

In FIG. 11A, real-time proliferation curves spanning 96 hours confirm that cells receiving the combination experience significantly diminished growth compared to single-drug or control groups. FIG. 11B illustrates colony formation assays in which treated cells (1×10\hat{ }3 or 3×10\hat{ }3) were seeded in six-well plates, grown for up to 14 days, and stained with crystal violet. The combined regimen yielded markedly fewer colonies than either cisplatin or aripiprazole alone, underscoring the potent cytotoxic synergy in pancreatic cancer cells.

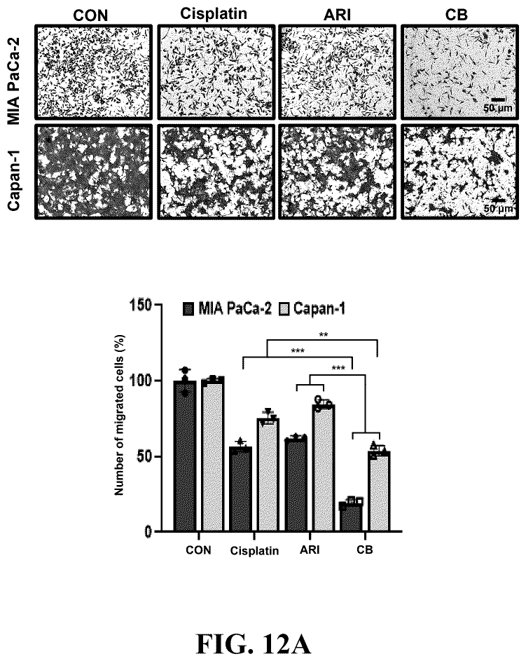

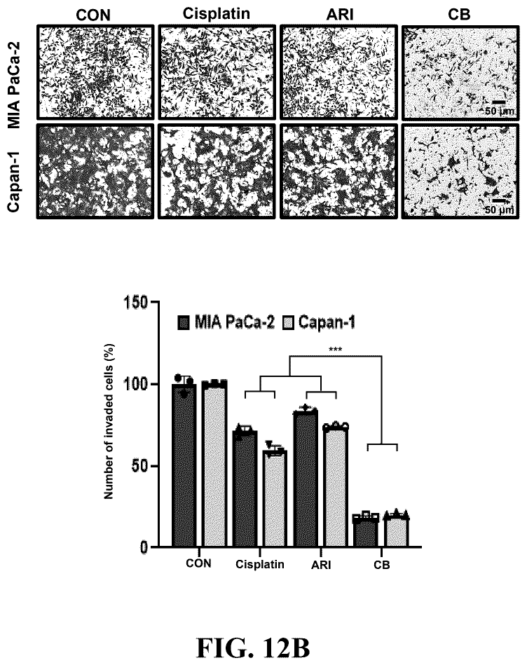

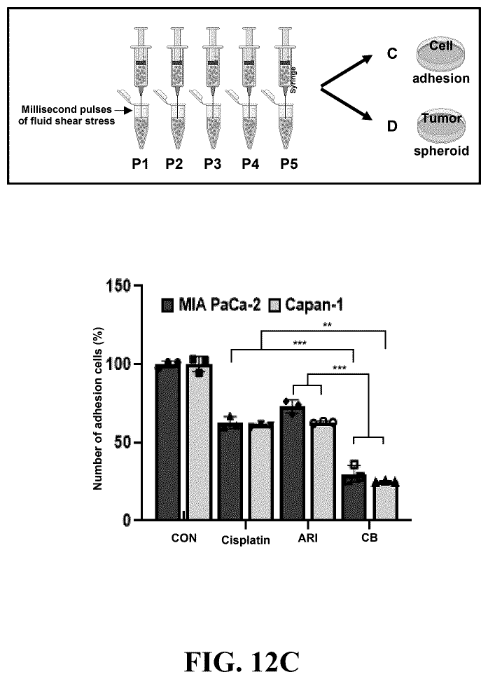

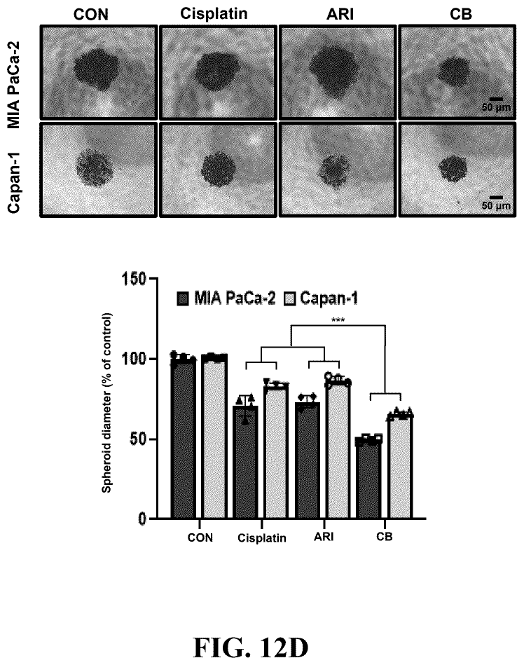

FIGS. 12A-12D demonstrate how these effects extend to metastatic properties of pancreatic cancer. FIG. 12A depicts Transwell migration assays without Matrigel, revealing that the combination significantly impedes cell motility. FIG. 12B presents invasion assays with Matrigel-coated inserts; once again, the dual treatment shows a stronger inhibitory effect than either agent alone. FIG. 12C illustrates a fluid shear stress setup in which pre-exposed cells travel through a high-shear needle system before being placed in ultra-low attachment plates. The combination disrupts cell adhesion and spheroid development under shear conditions, diminishing the potential for tumor progression. FIG. 12D displays final spheroids in 3D cultures, confirming that the combination yields smaller, less dense spheroids, an important observation for limiting metastasis in pancreatic cancer cells.

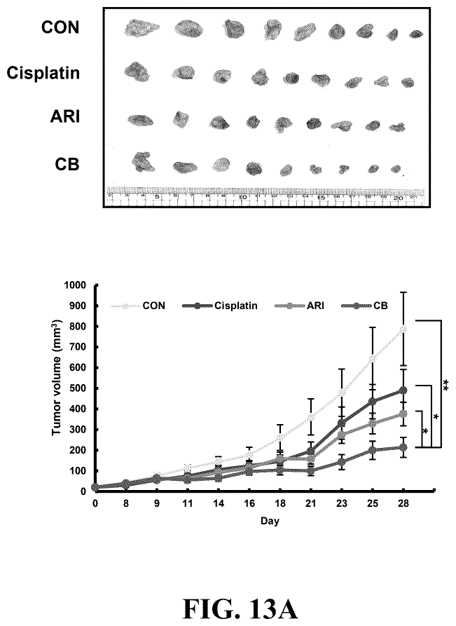

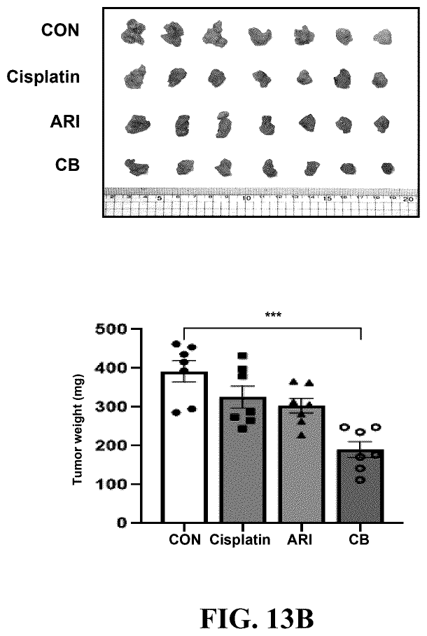

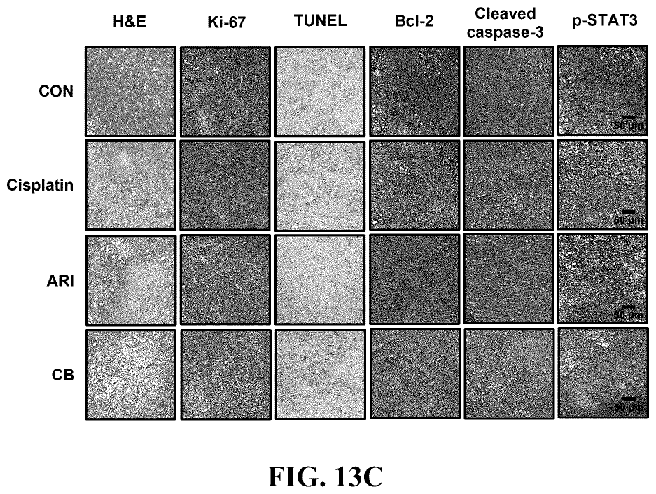

FIGS. 13A-13C illustrate the therapeutic relevance of combining aripiprazole with cisplatin in vivo. FIG. 13A shows representative tumors from a subcutaneous xenograft model in BALB/c nude mice bearing MIA PaCa-2 cells, with four groups: control (CON), cisplatin alone, aripiprazole (ARI) alone, and their combination (COMBI). Tumor volumes are significantly lower in the combination group than in any single-agent or control arm, underscoring a marked antitumor effect.

FIG. 13B presents comparable findings from an orthotopic KPC model of pancreatic cancer: after injecting 1×10\hat{ }4 KPC cells into the pancreas, mice received cisplatin (2 mg/kg, i.p., once weekly) and/or aripiprazole (10 mg/kg, p.o., thrice weekly) for 25 days. The final tumor weights reveal substantially greater reduction in the combination group compared with either agent alone, indicating robust synergy in an orthotopic setting.

FIG. 13C highlights hematoxylin and eosin staining and immunohistochemical detection analyses (H&E staining; Ki-67, TUNEL, Bcl-2, Cleaved caspase-3, and p-STAT3). The combination group shows lower proliferative markers (Ki-67, Bcl-2), higher apoptotic indices (TUNEL, cleaved caspase-3), and significantly reduced p-STAT3, all of which mirror the in vitro data and further confirm the enhanced efficacy of aripiprazole plus cisplatin for treating pancreatic cancer.

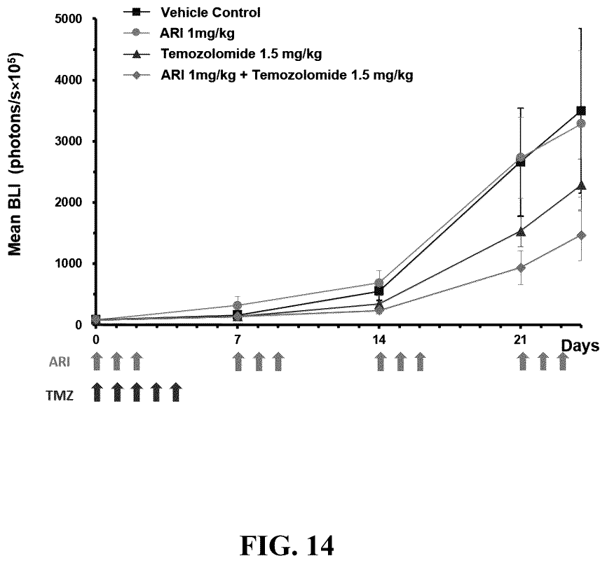

FIG. 14 highlights the evaluation of antitumor efficacy for various regimens in a U87-MG-luc orthotopic glioblastoma model, underscoring aripiprazole's broader potential beyond pancreatic cancer. In this study, Balb/c mice were anesthetized, and the intracranial region (cortex near the midline) was surgically exposed. A 2-μl suspension containing 2.5×10\hat{ }5 luciferase-expressing U87-MG cells was slowly injected into the right hemisphere. After closing the incision, the mice were randomized into four treatment arms, as detailed in Table 1:
Vehicle group (n=10), receiving only the base formulation orally, three days on/four days off, for four weeks.
Temozolomide group (1.5 mg/kg, n=10), administered once daily (q.d.) for five days.
Aripiprazole group (1 mg/kg, n=10), administered orally, three days on/four days off, for four weeks.
Combination group (aripiprazole 1 mg/kg plus temozolomide 1.5 mg/kg, n=10), following the schedules above for each agent.

Tumor progression was monitored weekly via bioluminescence imaging (BLI) using the IVIS Lumina III system. Regions of interest (ROIs) were quantified in photons/s to gauge tumor burden, with a BLI decrease (%) defined as 100×[1-(BLI_treatment/BLI_control)]. The mean BLI values at Day 24 (listed in Table 2) indicate that:
Vehicle-treated mice showed the highest tumor burden (3,496+1,341 photons/s×10\hat{ }5).
Temozolomide alone reduced BLI to 2,285+421 (34.6% decrease).
Aripiprazole alone yielded a modest effect, reducing BLI to 3,284+1,195 (6.1% decrease).
The dual regimen (aripiprazole+temozolomide) produced the most pronounced inhibition, with BLI values falling to 1,468+421 and a 58.0% decrease compared to vehicle.

This synergistic reduction in tumor-associated luminescence is shown in the line graph in FIG. 14, where the combination curve remains markedly lower than the curves for each single agent (aripiprazole alone, temozolomide alone) or the control. From Day 14 onward, tumor growth in the combination arm diverges sharply from the other groups, suggesting that aripiprazole meaningfully enhances temozolomide's efficacy in this orthotopic glioblastoma setting.

Notably, no additional clinical toxicity was observed during the study period. These findings reinforce the concept that aripiprazole can serve as an effective chemo-sensitizing agent with multiple anticancer therapeutics, here extending beyond cisplatin to include temozolomide for treating highly aggressive brain tumors. Consequently, FIG. 14 offers a strong translational implication for the repositioning of aripiprazole to boost tumor growth suppression even in notoriously resistant malignancies such as glioblastoma.

TABLE 1
The experimental groups and dosage

| Groups | Animals/group | Dose (mg/kg) | Route | Frequency | Duration | |
|---|---|---|---|---|---|---|
| Vehicle | 10 | — | P.O. | 3 days on/4 days off | 4 | weeks |
| Temozolomide | 10 | 1.5 | P.O. | qd | 5 | days |
| VS-101 | 10 | 1 | P.O. | 3 days on/4 days off | 4 | weeks |
| VS-101+ Temozolomide | 10 | 1 | P.O. | 3 days on/4 days off | 4 | weeks |
| | | 1.5 | P.O. | qd | 5 | days |

TABLE 2
Antitumor Activity in Different Treatment
Groups on Day 24 for Efficacy Study

| | Dose | Mean BLI Values | BLI Decrease |
|---|---|---|---|
| Groups | (mg/kg) | (photons/s × 10⁵) | (%) |
| Vehicle | — | 3,496 ± 1,341 | — |
| Temozolomide | 1.5 | 2,285 ± 421 | 34.6 |
| ARI | 1 | 3,284 ± 1,195 | 6.1 |
| ARI+ Temozolomide | 1 | | 58.0 |
| | 1.5 | 1,468 ± 421 | |

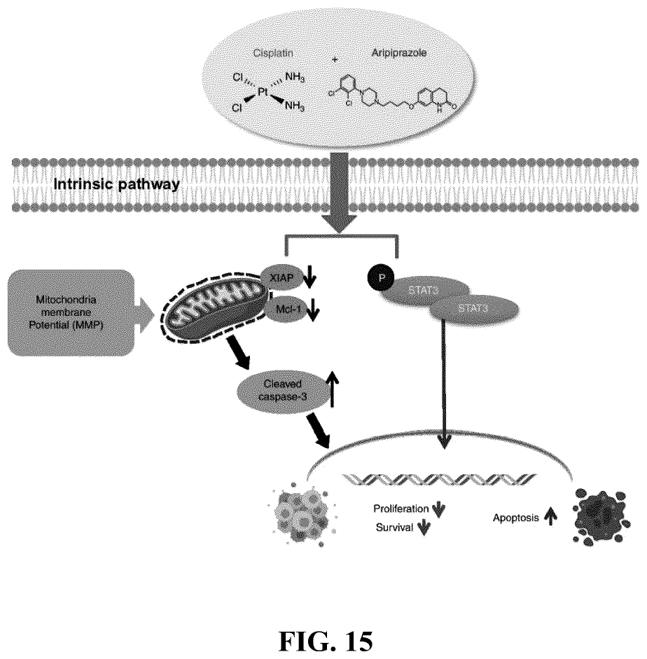

FIG. 15 offers a unifying schematic of how cisplatin and aripiprazole jointly disrupt tumor survival pathways. The combination provokes significant mitochondrial dysfunction (lowering membrane potential), decreases key anti-apoptotic proteins (XIAP and MCL-1), and inhibits STAT3-a multi-faceted transcription factor involved in tumor growth and resistance. By integrating these mechanisms, the dual therapy substantially boosts cleaved caspase-3 levels, reinforcing a powerful pro-apoptotic process. In doing so, it addresses a critical need in pancreatic cancer therapy: improving efficacy while tackling the high levels of chemoresistance and metastasis that characterize pancreatic cancer cells. Notably, the low toxicity profile of aripiprazole, originally approved as an antipsychotic, further emphasizes the advantages of drug repositioning, offering a novel and potentially more tolerable avenue to enhance platinum-based regimens and improve outcomes for patients with advanced cancers.

The experimental findings presented herein consistently demonstrate that aripiprazole, an antipsychotic compound originally approved for the treatment of psychiatric disorders, can act as a potent sensitizer when used in combination with various anticancer agents. Across multiple tumor cell lines (e.g., pancreatic, glioma, lung, and colorectal), the combination of aripiprazole and a chemotherapeutic (such as cisplatin or temozolomide) reduces cell proliferation more effectively than either agent alone. These effects are confirmed by multiple assays: MTT and CellTiter-Glo analyses reveal lower cell viability and synergistic combination indices (CI<1), while TUNEL assays, immunofluorescent staining of cleaved caspase-3, and JC-1 mitochondrial membrane potential measurements illustrate pronounced induction of apoptotic pathways.

Mechanistically, in some embodiments, the data show that administration of aripiprazole together with a platinum-based agent (or temozolomide) disrupts key molecular regulators of cell survival. Upregulation of cleaved caspase-3, downregulation of anti-apoptotic proteins (e.g., XIAP, MCL-1, and Bcl-2), and inhibition of the transcription factor STAT3 all converge to promote cancer cell death. Notably, phospho-kinase array studies reveal a broad reduction in signaling pathways associated with tumor proliferation, while Western blotting confirms significant decreases in p-STAT3 under combination therapy.

In vivo experiments in both subcutaneous and orthotopic tumor models align with the in vitro synergy findings. In a pancreatic cancer model, the combination of aripiprazole and cisplatin elicits substantial tumor shrinkage and an overall increase in apoptotic markers. Similarly, in a U87-MG-luc orthotopic glioblastoma model, co-treatment with aripiprazole and temozolomide yields a marked reduction in bioluminescent signals, reflecting diminished tumor burden. Importantly, the added therapeutic benefit from this combination appears to come without significant incremental toxicity.

Overall, this disclosure underscores the value of repositioning aripiprazole as a chemo-sensitizing agent. By lowering mitochondrial membrane potential, decreasing anti-apoptotic proteins, and curbing STAT3 activity, aripiprazole amplifies the tumoricidal effects of well-established anticancer therapies. These collective results suggest that a dual regimen involving aripiprazole and chemotherapeutics not only addresses the challenges of drug resistance and tumor aggressiveness but also offers a potentially more tolerable approach to managing advanced and hard-to-treat malignancies.

Further analyses revealed that aripiprazole's chemo-sensitizing influence extends beyond the modulation of STAT3 and intrinsic apoptotic pathways. In particular, a dual mechanism emerged: not only does aripiprazole suppress critical survival factors (e.g., cFLIP, MCL-1), but it also potentiates DNA damage signals in treated tumor cells. Such DNA damage augmentation, when combined with cFLIP downregulation, activates both extrinsic and intrinsic apoptotic programs, thereby intensifying the cancer cell kill. To elucidate these additional insights, the following section delves into DNA damage response (DDR) pathways and highlights how aripiprazole's synergy with DNA-damaging agents further drives malignant cells toward apoptotic demise.

### Mechanism Behind the Synergistic Cancer Treatment—DNA Damage Response

Cancer cells often harbor genetic instabilities that worsen when exposed to agents causing DNA damage. In many tumor types, unresolved DNA lesions can trigger apoptotic pathways, thereby halting further malignant progression. The figures below illustrate the major components of the DNA damage response (DDR), highlight the role of cFLIP in regulating extrinsic apoptosis, and demonstrate how chemotherapeutic or radiation-induced DNA breaks amplify cell death signaling.

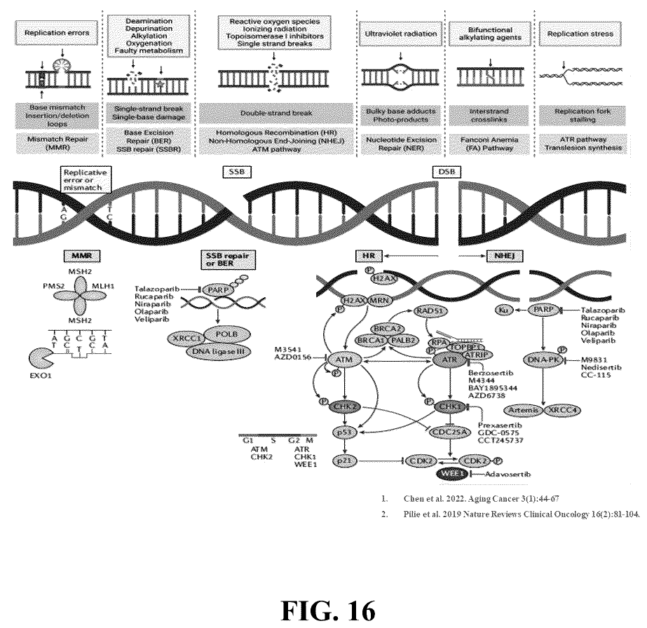

FIG. 16 provides an overview of the DNA damage response (DDR) that cells mount after experiencing double-strand breaks (DSBs) or single-strand breaks (SSBs). Various insults (e.g., chemical agents, ionizing radiation) can damage the DNA backbone, triggering distinct pathways such as homologous recombination (HR) or non-homologous end joining (NHEJ) for DSB repair, and base-excision repair (BER) for SSB repair. Key proteins-including ATM, ATR, DNA-PK, PARP, CHK1, and CHK2-become activated and orchestrate downstream responses to decide whether the cell repairs its DNA, pauses the cell cycle, or undergoes apoptosis if the damage proves too extensive. FIG. 16 also illustrates typical chemical and physical causes of DNA lesions-such as alkylating agents, topoisomerase inhibitors, and reactive oxygen species-and maps each class of DNA lesion to its corresponding repair or checkpoint pathway (adapted from Chen et al. 2022. Aging Cancer 3(1): 44-67 and Pilie et al. 2019. Nat Rev Clin Oncol 16(2): 81-104).

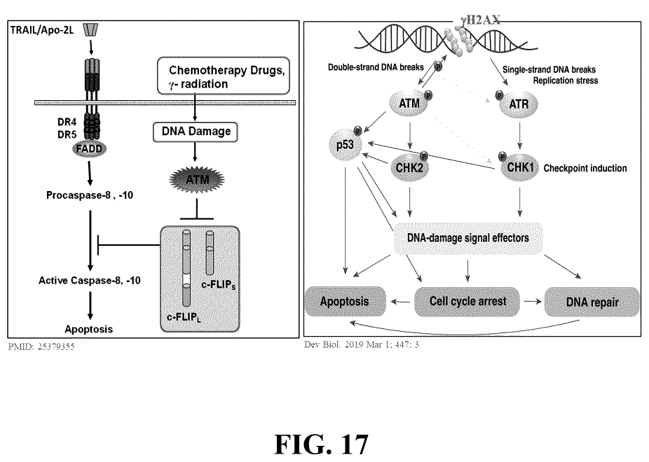

FIG. 17 highlights how DNA damage enhancement can be further amplified by chemotherapeutic agents or ionizing radiation, leading to heightened cell stress and increased γ-H2AX (a molecular marker of double-strand breaks). The left sub-diagram shows a simplified cascade where chemotherapy or γ-irradiation induces DNA breaks, activating ATM (and associated sensors), which in turn can modulate downstream effectors, including caspase-8/10 and cFLIP. The right sub-diagram offers a more detailed view of how ATM, ATR, and CHK1/2coordinate cell-fate decisions—either cell cycle arrest, DNA repair, or apoptosis—in response to accumulating DNA damage signals. The illustrated γ-H2AX foci mark sites of DSBs, confirming that DNA fragmentation has occurred and signaling the cell to attempt repair; if unsuccessful or overwhelmed, the cell transitions into apoptosis (adapted in part from Dev Biol. 2019 Mar. 1;447:3 and PMID: 25379355).

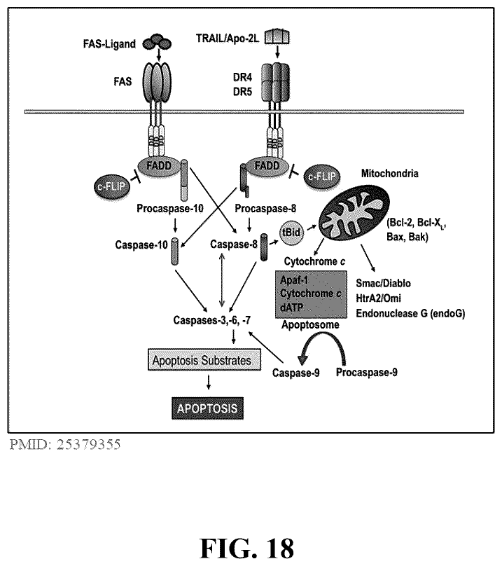

Building on the foundational understanding of DDR, FIG. 18 schematically depicts how cFLIP inhibition, following DNA damage enhancement, promotes extrinsic apoptosis in tumor cells. The cell-surface receptors Fas (or TRAIL/Apo2L receptors DR4/DR5) bind to their corresponding ligands, initiating a death signal through FADD (Fas-associated protein with death domain). Under normal conditions, cFLIP (cellular FLICE-like inhibitory protein) suppresses the activation of caspase-8 (and caspase-10), thereby inhibiting the extrinsic apoptosis pathway. By contrast, when cFLIP is downregulated or functionally blocked, the extrinsic pathway proceeds unhindered, enabling robust caspase-8 activation and subsequent cleavage of downstream effector caspases (e.g., caspases-3, -6, -7). In parallel, t-Bid generated by caspase-8 can act on mitochondria to trigger the intrinsic (mitochondrial) apoptotic pathway, releasing cytochrome c and other pro-apoptotic factors (e.g., Smac/DIABLO). The convergence of extrinsic and intrinsic pathways culminates in strong apoptotic cell death. The figure underscores how enhanced DNA damage plus cFLIP inhibition acts as a dual mechanism to push cancer cells toward apoptosis more effectively (adapted from PMID: 25379355).

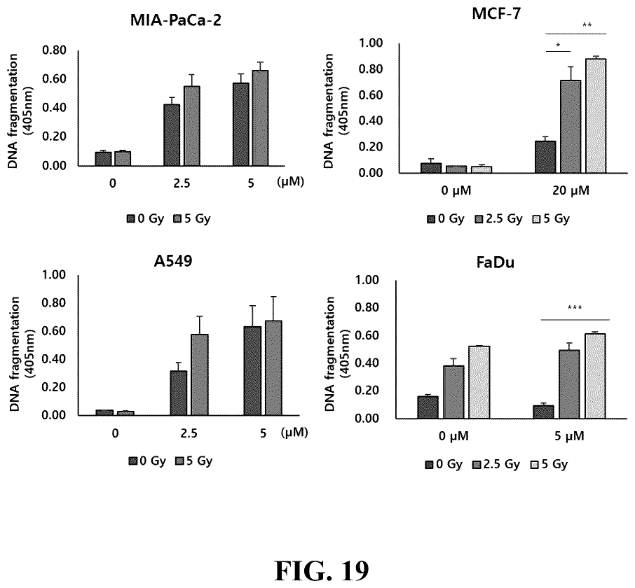

The following figures (FIGS. 19-26) present a series of experiments demonstrating how aripiprazole, in combination with various DNA-damaging stimuli (e.g., γ-irradiation), leads to enhanced DNA fragmentation, prolonged DNA damage signaling, and cFLIP downregulation across multiple tumor cell lines. Taken together, these data reveal that by potentiating the DNA damage response (DDR) and suppressing cFLIP, aripiprazole promotes both extrinsic and intrinsic apoptotic pathways in a broad range of cancers (including breast, pancreatic, prostate, lung, and head and neck malignancies). Although the primary DNA damage source in these studies is γ-irradiation, similar mechanisms are expected for other DNA-damaging agents, such as alkylating drugs. The results support the notion that cFLIP inhibition, coupled with heightened DNA damage, can synergistically push cancer cells toward apoptosis, thereby offering a promising therapeutic strategy.

FIG. 19 illustrates the induction of DNA fragmentation across four different cancer cell lines (MIA-PaCa-2, MCF-7, A549, and FaDu) upon treatment with aripiprazole and/or ionizing radiation (IR). The ELISA-based DNA Fragmentation Assay was carried out after 48 hours of incubation. Bars indicate measured optical density (OD) at 405 nm, reflecting the extent of DNA fragmentation. In each panel, doses of aripiprazole (e.g., 2.5 μM, 5 μM) alone and in combination with incremental radiation doses (0 Gy, 2.5 Gy, 5 Gy) show that co-treatment generally yields higher levels of DNA fragmentation compared to either agent alone. This finding supports the premise that aripiprazole can potentiate radiation-induced DNA damage, thereby amplifying cell death signals in a variety of tumor models.

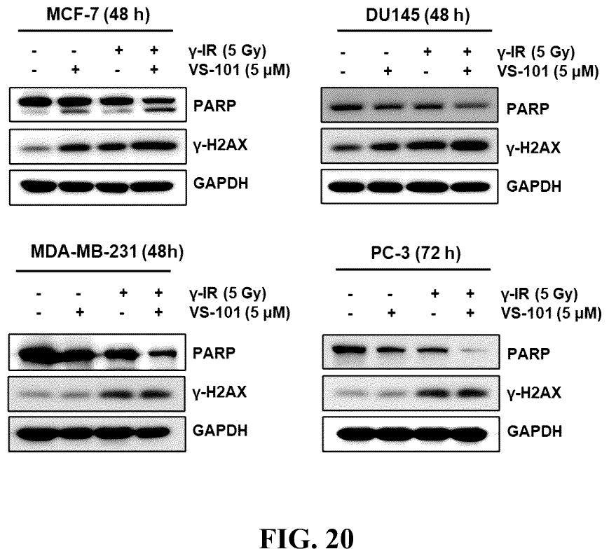

Turning to FIG. 20, multiple Western blots demonstrate “sustained DNA damage response” upon aripiprazole treatment, either alone or in combination with γ-IR (5 Gy). The cell lines assessed include MCF-7 (breast cancer), DU145 (prostate cancer), MDA-MB-231 (breast cancer), and PC-3 (prostate cancer), at various time points (48 hours or 72 hours). PARP cleavage (PARP) and γ-H2AX (a biomarker for DNA double-strand breaks) are probed, with GAPDH serving as a loading control. In the left panels, cells receiving both aripiprazole and γ-IR show elevated γ-H2AX and altered PARP profiles compared to controls, indicating heightened or prolonged DNA damage. The right-side sub-panels consolidate these findings by highlighting that aripiprazole alone can induce γ-H2AX but exhibits an even more pronounced effect when combined with radiation, reinforcing the notion that aripiprazole enhances or sustains DNA damage signaling in diverse tumor types.

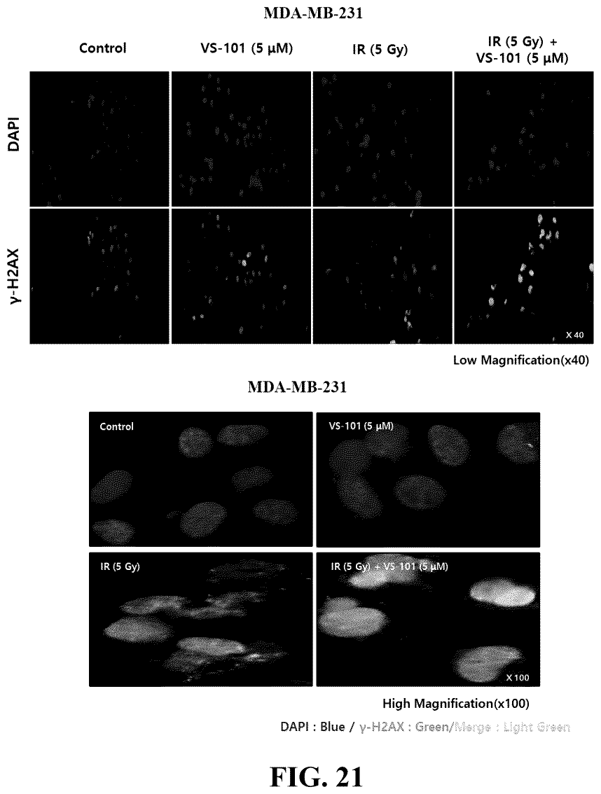

FIG. 21 employs immunocytochemistry (ICC) staining to visualize γ-H2AX foci in the MDA-MB-231 breast cancer cell line under four conditions: (i) Control, (ii) aripiprazole alone (5 μM), (iii) IR alone (5 Gy), and (iv) a combination of IR plus aripiprazole. The nuclei are counterstained with DAPI (blue), while γ-H2AX is shown in green. At low magnification (×40) above, an increased number of green γ-H2AX foci is evident in treated groups, particularly in the combination group, suggesting enhanced DNA double-strand breaks. The high-magnification images (×100) below provide a more detailed view of γ-H2AX accumulation within the nucleus. This ICC analysis further corroborates that co-treatment of aripiprazole with radiation yields greater DNA damage than either treatment alone, as evidenced by robust γ-H2AX induction.

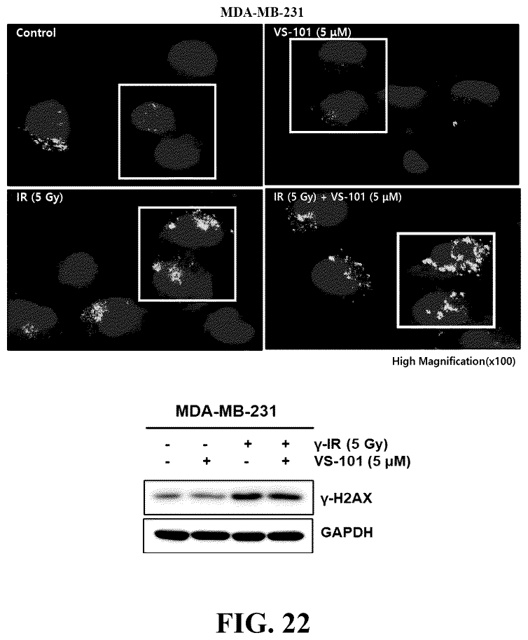

Building on the foregoing evidence, FIG. 22 focuses again on MDA-MB-231 cells, with a slight variation in experimental conditions (24 hours incubation; ICC methods for γ-H2AX, and a corresponding Western blot). Panel images (top) show representative immunofluorescence fields at high magnification (×100). Boxes highlight key nuclear regions with extensive γ-H2AX (green) puncta. The Western blot (bottom) compares γ-H2AX expression under different treatment regimens: control (untreated), aripiprazole (5 μM), γ-IR (5 Gy), and their combination. As in FIG. 21, the dual treatment group (aripiprazole+γ-IR) exhibits the highest γ-H2AX signal, confirming that aripiprazole significantly augments radiation-induced DNA damage in MDA-MB-231 cells. Overall, FIG. 22 reinforces the conclusion that aripiprazole intensifies or prolongs DNA damage responses, thereby potentiating apoptotic cell death in human breast cancer models.

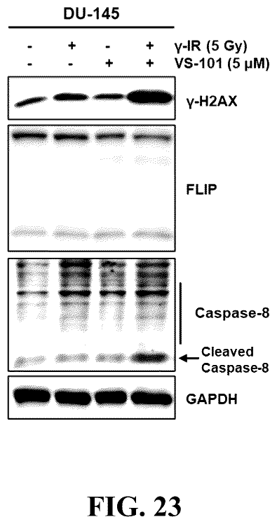

FIG. 23 demonstrates how cFLIP inhibition can steer tumor cells toward extrinsic apoptosis, as evidenced by changes in γ-H2AX, FLIP, and Caspase-8. DU-145 (prostate cancer) was treated with γ-irradiation (5 Gy), aripiprazole (5 μM), or a combination thereof for 48 hours. Western blot analysis shows an increase in γ-H2AX (indicating DNA damage) alongside a reduction in cFLIP levels (both L and/or S isoforms), concomitant with Caspase-8 activation (cleaved form). These results support the conclusion that aripiprazole, when combined with DNA-damaging stimuli, lowers cFLIP and thereby unleashes the extrinsic apoptotic pathway.

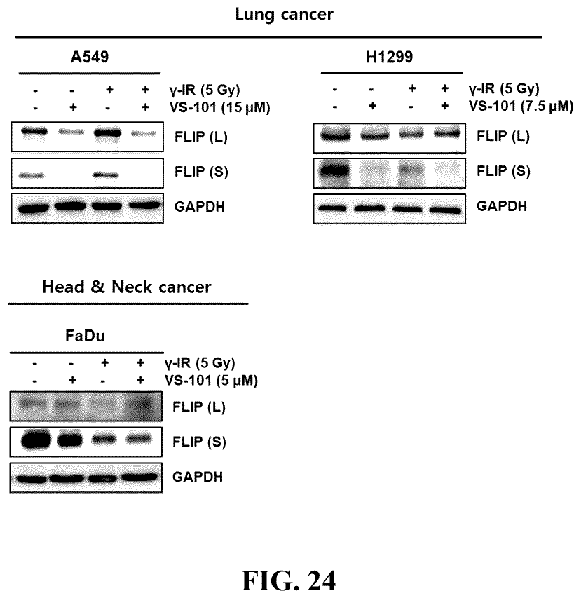

FIG. 24 focuses on the downregulation of cFLIP in lung cancer (A549, H1299) and head and neck cancer (FaDu) cells following treatment with aripiprazole and γ-irradiation (5 Gy). Western blots track both cFLIP (L) and cFLIP(S) isoforms, along with a GAPDH loading control. Notably, in each cell line, aripiprazole plus IR more profoundly reduces cFLIP expression compared to either agent alone, reinforcing the idea that heightened DNA damage leads to cFLIP depletion. This observation aligns with the earlier hypothesis that suppressing cFLIP removes a key block on caspase activation, thereby sensitizing cancer cells to extrinsic apoptosis signals.

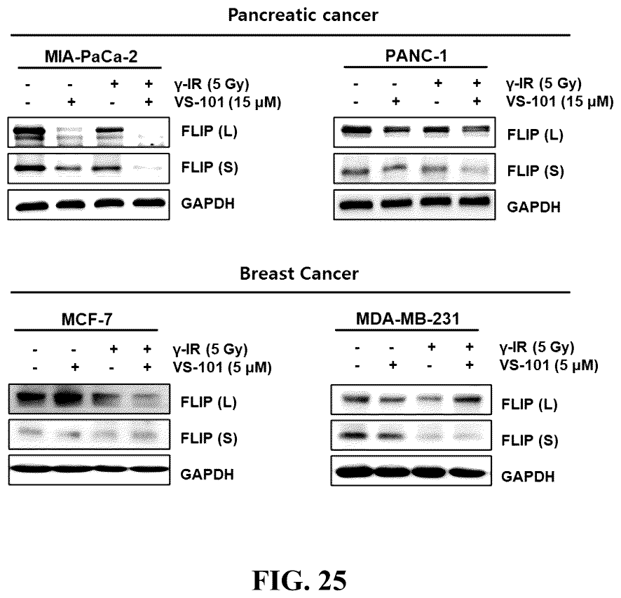

Turning to pancreatic (MIA-PaCa-2, PANC-1) and breast (MCF-7, MDA-MB-231) cancer cells, FIG. 25 again highlights FLIP (L) and FLIP(S) regulation under γ-IR (5 Gy) and aripiprazole treatment (various concentrations). In both tumor types, Western blot results confirm that combination treatment diminishes both FLIP isoforms relative to control or single-agent conditions. GAPDH is included as a loading control. These data suggest that pancreatic and breast cancer cells share a common mechanism whereby aripiprazole-induced DNA damage correlates with cFLIP suppression, ultimately facilitating apoptotic cascades in diverse malignancies.

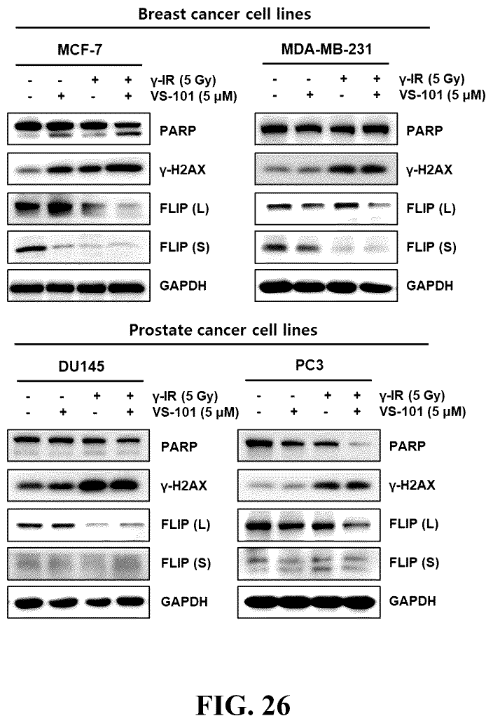

Finally, FIG. 26 provides a confirmation of protein expression in breast (MCF-7,MDA-MB-231) and prostate (DU145, PC3) cancer cell lines following γ-IR (5 Gy) and aripiprazole (5 μM) exposure. The Western blot panels illustrate elevated PARP cleavage and γ-H2AX, accompanied by lower FLIP (L) and FLIP(S). GAPDH is used as an internal standard. Across all four cell lines, the co-treatment group exhibits the most robust changes, signifying that aripiprazole combined with ionizing radiation consistently drives DNA damage and cFLIP downregulation, thereby augmenting extrinsic apoptosis in multiple cancer types.

In the above experiments, γ-irradiation has been employed as the primary means of inducing DNA damage, and no separate experiments have yet been conducted using chemical agents (e.g., alkylating agents, topoisomerase inhibitors) to produce such lesions. However, because both ionizing radiation and chemical DNA-damaging therapies ultimately generate single-strand breaks (SSBs) or double-strand breaks (DSBs)—thereby activating similar downstream repair and apoptotic pathways—it is anticipated that comparable results would likely be obtained if chemical DNA-damaging agents were tested in conjunction with aripiprazole. In particular, therapies based on platinum coordination complexes (e.g., cisplatin, carboplatin) or alkylating drugs (e.g., temozolomide, cyclophosphamide) commonly induce DSBs or persistent DNA adducts that lead to apoptosis when left unrepaired. Given the data demonstrating that aripiprazole lowers cFLIP and heightens DDR signals in irradiated cells, it is reasonable to expect a similar chemo-sensitizing effect against DNA-damaging chemical agents. As such, the mechanism of synergy—i.e., reinforcing extrinsic and intrinsic apoptotic pathways in response to heightened DNA damage—supports the conclusion that aripiprazole would exhibit analogous efficacy in combination with other DNA-damaging regimens.

It should be understood that variations, clarifications, or modifications are contemplated. Applications of the technology to other fields not mentioned are also contemplated.

Exemplary methods and compositions are described. Since numerous modifications and changes will readily be apparent to those having ordinary skill in the art, it is not desired to limit the invention to only the exact constructions as demonstrated in this disclosure. Accordingly, all suitable modifications and equivalents may be resorted to falling within the scope of the invention.

It should be understood that claims that include fewer limitations, broader claims, such as claims without requiring a certain feature or process step in the appended claim or in the specification, clarifications to the claim elements, different combinations, and alternative implementations based on the specification, or different uses, are also contemplated by the embodiments of the present invention.

It should be understood that combinations of described features or steps are contemplated even if they are not described directly together or not in the same context.

The terms or words that are used herein are directed to those of ordinary skill in the art in this field of technology and the meaning of those terms or words will be understood from terminology used in that field or can be reasonably interpreted based on the plain English meaning of the words in conjunction with knowledge in this field of technology. This includes an understanding of implicit features that for example may involve multiple possibilities, but to a person of ordinary skill in the art a reasonable or primary understanding or meaning is understood.

Unless defined otherwise, all technical and scientific terms used herein have same meaning as commonly understood by the person of ordinary skill in the art to which this invention belongs.

It should be understood that the above description of the invention and specific examples, while indicating preferred embodiments of the present invention, are given by way of illustration and not limitation. Many changes and modifications within the scope of the present invention may be made without departing from the spirit thereof, and the present invention includes all such changes and modifications.

What is claimed is:

1. A method of treating cancer in a patient in need thereof, the method comprising:
conjointly administering aripiprazole and an anticancer agent to the patient, thereby treating the cancer.

2. The method of claim 1, wherein conjointly administering aripiprazole and the anticancer agent provides a synergistic effect.

3. The method of claim 1, wherein the cancer is selected from the group consisting of lung cancer, colorectal cancer, pancreatic cancer, and glioma.

4. The method of claim 1, wherein the anticancer agent is selected from the group consisting of cisplatin, carboplatin, oxaliplatin, gemcitabine, 5-FU, and temozolomide.

5. The method of claim 1, wherein the anticancer agent is cisplatin.

6. A method of treating pancreatic cancer in a patient in need thereof, the method comprising:
conjointly administering aripiprazole and an anticancer agent to the patient, thereby treating the pancreatic cancer.

7. The method of claim 6, wherein conjointly administering aripiprazole and the anticancer agent provides a synergistic effect.

8. The method of claim 6, wherein the anticancer agent is selected from the group consisting of cisplatin, carboplatin, oxaliplatin, gemcitabine, 5-FU, and temozolomide.

9. The method of claim 6, wherein the anticancer agent is cisplatin.

10. A method of treating glioma in a patient in need thereof, the method comprising:
conjointly administering aripiprazole and an anticancer agent to the patient, thereby treating the glioma.

11. The method of claim 10, wherein conjointly administering aripiprazole and the anticancer agent provides a synergistic effect.

12. The method of claim 10, wherein the anticancer agent is selected from the group consisting of cisplatin, carboplatin, oxaliplatin, gemcitabine, 5-FU, and temozolomide.

13. The method of claim 10, wherein the anticancer agent is cisplatin.

14. A method of treating colorectal cancer in a patient in need thereof, the method comprising:
conjointly administering aripiprazole and an anticancer agent to the patient, thereby treating the colorectal cancer.

15. The method of claim 14, wherein conjointly administering aripiprazole and the anticancer agent provides a synergistic effect.

16. The method of claim 14, wherein the anticancer agent is selected from the group consisting of cisplatin, carboplatin, oxaliplatin, gemcitabine, 5-FU, and temozolomide.

17. The method of claim 14, wherein the anticancer agent is carboplatin.

18. A method of treating lung cancer in a patient in need thereof, the method comprising:
conjointly administering aripiprazole and an anticancer agent to the patient, thereby treating the lung cancer.

19. The method of claim 18, wherein conjointly administering aripiprazole and the anticancer agent provides a synergistic effect.

20. The method of claim 18, wherein the anticancer agent is selected from the group consisting of cisplatin, carboplatin, oxaliplatin, gemcitabine, 5-FU, and temozolomide.
